(19) United States
(12) Patent Application Publication
(10) Pub. No.: US 2024/0096448 A1
(43) Pub. Date: Mar. 21, 2024

(54) COMPUTER-IMPLEMENTED METHOD FOR PREPARING OLIGONUCLEOTIDES USED TO DETECT NUCLEOTIDE MUTATION OF INTEREST

(71) Applicant: SEEGENE, INC., Seoul (KR)

(72) Inventors: Hyun Ho KIM, Seoul (KR); Daeyoung KIM, Seoul (KR); Giseok YOON, Seoul (KR); Na Young SHIN, Gyeonggi-do (KR); Hyun Ju RYOO, Seoul (KR)

(21) Appl. No.: 18/038,650

(22) PCT Filed: Dec. 10, 2021
(86) PCT No.: PCT/KR2021/018746

§ 371 Date: May 24, 2023

(30) Foreign Application Priority Data
Dec. 11, 2020 (KR) 10-2020-0173648

(51) Int. Cl. G16B 25/20; G16B 20/20; G16B 30/10

(52) U.S. Cl. CPC G16B 25/20; G16B 20/20; G16B 30/10

(57) ABSTRACT

The present invention relates to a computer-implemented method for preparing oligonucleotides used to detect a nucleotide mutation of interest in a target nucleic acid sequence. The present invention can provide oligonucleotides capable of detecting a nucleotide mutation on the basis of an integrated design rule, without the need to develop detailed design rules and modules considering the type of nucleotide mutation, the size of the mutation, whether a target to be detected is a wild-type and/or mutant sequence, sequence contents, and the like, by inputting information about a wild-type target nucleic acid sequence and a nucleotide mutation of interest, providing wild-type and mutant target nucleic acid sequences through the use of the input information, designing oligonucleotides for the mutant target nucleic acid sequence, and analyzing the matching between the designed oligonucleotides and the wild-type target nucleic acid sequence to select and provide oligonucleotides satisfying predetermined selection criteria.

### CROSS-REFERENCE TO RELATED APPLICATIONS

This application claims priority from Korean Patent Application No. 2020-0173648, filed on Dec. 11, 2020 in the Korean Intellectual Property Office, the disclosure of which is incorporated herein by reference.

### BACKGROUND OF THE INVENTION

### Field of the Invention

The present invention relates to a computer-implemented method for preparing oligonucleotides used to detect a nucleotide mutation of interest in a target nucleic acid sequence.

### Description of the Related Art

Many methods for detecting target nucleic acid sequences are known in the art. Examples of the various detection methods include the following: PTO cleavage and extension (PTOCE) method (WO 2012/096523), TaqMan probe method (U.S. Pat. No. 5,210,015), Molecular beacon method (Tyagi et al., Nature Biotechnology 14:303 (1996)), Scorpion method (Whitcombe et al., Nature Biotechnology 17:804-807 (1999)), Sunrise or Amplifluor method (Nazarenko et al., 2516-2521 Nucleic Acids Research, 25(12):2516-2521 (1997), and U.S. Pat. No. 6,117,635), Lux method (U.S. Pat. No. 7,537,886), CPT (Duck P, et al., Biotechniques, 9:142-148 (1990)), LNA method (U.S. Pat. No. 6,977,295), Plexor Method (Sherrill C B, et al., Journal of the American Chemical Society, 126:4550-4556(2004)), Hybeacons method (D. J French, et al., Molecular and Cellular Probes (2001) 13, 363-374 and U.S. Pat. No. 7,348,141), a dual-labeled, self-quenched probe (U.S. Pat. No. 5,876,930), a hybridization probe (Bernard P S, et al., Clin Chem 2000, 46, 147-148), Invader analysis (U.S. Pat. No. 5,691,142), PTO Cleavage and Extension-Dependent Signaling Oligonucleotide Hybridization (PCE-SH) method (WO 2013/115442), PTO Cleavage and Extension-Dependent Non-Hybridization (PCE-NH) method (WO 2014/104818), and CER method (WO 2011/037306).

Oligonucleotides (probes and/or primers) used for the detection of target nucleic acid sequences must have suitable specificity and sensitivity, and are suitable for certain detection methods and need to meet the conditions set by the analysts. It is therefore very important to design oligonucleotides suitable for the purpose of analysis.

Meanwhile, nucleotide mutations including substitution (e.g., single nucleotide polymorphism (SNP)), inversion, insertion, deletion, duplication, or a complex thereof are important in the research and clinical areas. SNPs are most frequently found in the human genome and serve as a marker for disease-associated localization and pharmacogenetics. SNPs are found at a rate of approximately 1 per 1,000 bp in the human genome, and the total number thereof is estimated to be about 3 million.

Conventionally, the type of nucleotide mutation (e.g., substitution, insertion, etc.), the size of mutation (e.g., the number of mutant bases), and whether a target to be detected is a wild-type and/or mutant sequence had to be considered in order to design oligonucleotides for the detection of nucleotide mutations. As a result, there was a problem that at least 20 design rules and module needed to be developed according to these considerations.

Moreover, the conventional methods had a problem in that sequence contents were difficult to consider. For example, in cases where a wild-type target nucleic acid sequence is 5′-CAGATCTGTTTTAAACGACT-3′ (SEQ ID NO: 1), a mutation of inserting the base T occurs between the 11th and 12th positions from the 5′-end of the wild-type target nucleic acid sequence (i.e., a mutant target nucleic acid sequence of 5′-CAGATCTGTTTTTAAACGACT-3′ (SEQ ID NO:2)), and a design rule with respect to an insertion mutation is set that designs an oligonucleotide so as to locate a base for the insertion mutation at the third position from the 5′-end of the oligonucleotide (i.e., 5′-TTTTAAACGACT-3′ (SEQ ID NO:3)), an oligonucleotide designed according to the design rule cannot distinguishably detect between the wild-type target nucleic acid sequence and the mutant target nucleic acid sequence.

Therefore, the present inventors endeavored to develop an integrated design method capable of designing oligonucleotides used to distinguishably detect between wild-type and mutant target nucleic acid sequences considering all of various nucleotide mutation types.

Throughout this application, various patents and publications are referenced and citations are provided in parentheses. The disclosure of these patents and publications in their entities are hereby incorporated by references into this application in order to more fully describe this invention and the state of the art to which this invention pertains.

### SUMMARY OF THE INVENTION

The present inventors have endeavored to overcome the above-described problems of the conventional art and to develop a computer-implemented method capable of effectively preparing oligonucleotides used to detect a nucleotide mutation of interest in a target nucleic acid sequence. As a result, the present inventors confirmed that it is possible to prepare oligonucleotides capable of detecting a nucleotide mutation on the basis of an integrated design rule, without the need to develop detailed design rules and modules considering the type of nucleotide mutation, the size of the mutation, whether a target to be detected is a wild-type and/or mutant sequence, sequence contents, and the like, by inputting information about a wild-type target nucleic acid sequence and a nucleotide mutation of interest, providing wild-type and mutant target nucleic acid sequences through the use of the input information, designing oligonucleotides for the mutant target nucleic acid sequence, and analyzing the matching between the designed oligonucleotides and the wild-type target nucleic acid sequence to select and prepare oligonucleotides satisfying predetermined selection criteria, and thus the present inventors completed the present invention.

Accordingly, it is an object of the present invention to provide a computer-implemented method for preparing oligonucleotides used to detect a nucleotide mutation of interest in a target nucleic acid sequence.

It is another object of the present invention to provide a computer-readable storage medium including instructions to implement a process to perform a method for preparing oligonucleotides used to detect a nucleotide mutation of interest in a target nucleic acid sequence.

Other objects and advantages of the present invention will become apparent from the detailed description below taken in conjugation with the appended claims and drawings.

### BRIEF DESCRIPTION OF THE DRAWINGS

FIG. 1 is a flowchart showing a process of preparing oligonucleotides used to detect a nucleotide mutation of interest within a target nucleic acid sequence according to an embodiment of the present invention.

FIG. 2 shows a user interface (UI) for inputting information about a wild-type target nucleic acid sequence and a nucleotide mutation of interest according to an embodiment of the present invention.

### DETAILED DESCRIPTION OF THIS INVENTION

In an aspect of the present invention, there is provided a computer-implemented method for preparing an oligonucleotide used to detect a nucleotide mutation of interest in a target nucleic acid sequence, comprising:
(a) inputting information about a wild-type target nucleic acid sequence and a nucleotide mutation of interest; wherein the wild-type target nucleic acid sequence is a target nucleic acid sequence not comprising the nucleotide mutation of interest, the information includes information about (i) the wild-type target nucleic acid sequence, (ii) a position of the nucleotide mutation of interest occurring in the wild-type target nucleic acid sequence and (iii) wild-type and mutant bases at the position of the nucleotide mutation of interest, and wherein the position of the nucleotide mutation of interest is expressed as a start position and an end position,
(b) providing the wild-type target nucleic acid sequence and a mutant target nucleic acid sequence by using the input information; wherein the mutant target nucleic acid sequence is a target nucleic acid sequence comprising the nucleotide mutation of interest, and the wild-type and mutant target nucleic acid sequences include forward and reverse wild-type and mutant target nucleic acid sequences, respectively,
(c) providing a first oligonucleotide candidate group for the mutant target nucleic acid sequence by designing oligonucleotides used to detect the nucleotide mutation of interest in a predetermined region comprising the nucleotide mutation of interest within the mutant target nucleic acid sequence;
(d) providing oligonucleotides satisfying the following selection criteria as a second oligonucleotide candidate group for the mutant target nucleic acid sequence by analyzing matching of the wild-type target nucleic acid sequence with the oligonucleotides included in the first oligonucleotide candidate group for the mutant target nucleic acid sequence; wherein the selection criteria include that (i) the number of mismatches between the wild-type target nucleic acid sequence and a predetermined region at the 5′-end, middle or 3′-end of an oligonucleotide is one or more; or (ii) the ratio of mismatches between the wild-type target nucleic acid sequence and an oligonucleotide is a predetermined value or more; and
(e) providing a third oligonucleotide candidate group by selecting oligonucleotides from the second oligonucleotide candidate group for the mutant target nucleic acid sequence; wherein the third oligonucleotide candidate group is used to detect the nucleotide mutation of interest in the target nucleic acid sequence.

The present inventors have endeavored to overcome the above-described problems of the conventional art and to develop a computer-implemented method capable of effectively preparing oligonucleotides used to detect a nucleotide mutation of interest in a target nucleic acid sequence. As a result, the present inventors confirmed that it is possible to prepare oligonucleotides capable of detecting a nucleotide mutation on the basis of an integrated design rule, without the need to develop detailed design rules and modules considering the type of nucleotide mutation, the size of the mutation, whether a target to be detected is a wild-type and/or mutant sequence, sequence contents, and the like, by inputting information about a wild-type target nucleic acid sequence and a nucleotide mutation of interest, providing wild-type and mutant target nucleic acid sequences through the use of the input information, designing oligonucleotides for the mutant target nucleic acid sequence, and analyzing the matching between the designed oligonucleotides and the wild-type target nucleic acid sequence to select and prepare oligonucleotides satisfying predetermined selection criteria.

As used herein while reciting an oligonucleotide, the term “preparing” includes the providing of sequence information of an oligonucleotide and the manufacture of an oligonucleotide substance.

The term used herein, “target nucleic acid sequence” or “target sequence” refers to a particular nucleic acid sequence representing a target nucleic acid molecule.

As used herein, the term “target nucleic acid molecule”, “target molecule”, or “target nucleic acid” refers to a nucleotide molecule in an organism to be detected. A target nucleic acid molecule is generally given a particular name, and includes the whole genome and all nucleotide molecules constituting the genome (e.g., genes, pseudogenes, non-coding sequence molecules, untranslated region, and some regions of the genome). An example of the target nucleic acid molecule includes a nucleic acid of an organism.

As used herein, the term “organism” refers to an organism that belongs to the biological classification system, for example, kingdom, division, class, order, family, genus, species, subspecies, varieties, variant, subtype, genotype, serotype, strain, isolate or cultivar. Examples of the organism include prokaryotic cells (e.g., Mycoplasma pneumoniae, Chlamydophila pneumoniae, Legionella pneumophila, Haemophilus influenzae, Streptococcus pneumoniae, Bordetella pertussis, Bordetella parapertussis, Neisseria meningitidis; Listeria monocytogenes, Streptococcus agalactiae, Campylobacter; Clostridium difficile, Clostridium perfringens, Salmonella, Escherichia coli, Shigella, Vibrio, Yersinia enterocolitica, Aeromonas, Chlamydia trachomatis, Neisseria gonorrhoeae, Trichomonas vaginalis, Mycoplasma hominis Mycoplasma genitalium, Ureaplasma urealyticum, Ureaplasma parvum, Mycobacterium tuberculosis), eukaryotic cells (e.g., protozoa and parasites, fungi, yeast, higher plants, lower animals, and higher animals including mammals and humans), viruses, or viroids. Examples of the parasites in the eukaryotic cells include Giardia lamblia, Entamoeba histolytica, Cryptosporidium, Blastocystis hominis, Dientamoeba fragi/is, and Cyclospora cayetanensis Examples of the viruses are influenza A virus (Flu A), influenza B virus (Flu B), respiratory syncytial virus A (RSV A), respiratory syncytial virus B (RSV B), parainfluenza virus 1 (PIV 1), parainfluenza virus 2 (PIV 2), parainfluenza virus 3 (PIV 3), parainfluenza virus 4 (PIV 4), metapneumovirus (MPV), human enterovirus (HEV), human bocavirus (HBoV), human rhinovirus (HRV), coronavirus, and adenovirus, which cause respiratory diseases; and noroviruses, rotaviruses, adenoviruses, astroviruses, and sapoviruses that cause gastrointestinal diseases. Examples of the viruses include human papillomavirus (HPV), Middle East respiratory syndrome-related coronavirus (MERS-CoV), dengue virus, herpes simplex virus (HSV), human herpes virus (HHV), Epstein-Barr virus (EMV), varicella zoster virus (VZV), cytomegalovirus (CMV), HIV, hepatitis virus, and poliovirus.

As used herein, the term “nucleotide mutation of interest” refers to a nucleotide mutation in a target nucleic acid sequence to be amplified and/or detected using oligonucleotides (e.g., primers or probes).

As used herein, the term “nucleotide mutation” refers to any single or multiple nucleotide substitutions, deletions, inversions, insertions, deletions, duplications, or a complex thereof in a DNA sequence at a particular location among consecutive DNA segments or DNA segments that are otherwise similar in sequence. These contiguous DNA fragments include any other portion of one gene or one chromosome. Such nucleotide mutations may be mutant or polymorphic allele variations. For example, the nucleotide mutations include single nucleotide polymorphisms (SNPs), mutations, deletions, insertions, substitutions, and translocations, and also include numerous variations (e.g., variations in the methylenetetrahydrofolate reductase (MTHFR) gene) in a human genome, variations involved in drug resistance of pathogens, and tumorigenesis-causing variations.

According to an embodiment of the present invention, the nucleotide mutation of interest is a substitution, an inversion, an insertion, a deletion, a duplication, or a combination thereof.

As used herein, the term “oligonucleotide” refers to a linear oligomer of natural or modified monomers or linkages. The oligonucleotide includes deoxyribonucleotides and ribonucleotides, can specifically hybridizes with a target nucleotide sequence, and is naturally present or artificially synthesized. The oligonucleotide is especially a single chain for maximum efficiency in hybridization. Specifically, the oligonucleotide is an oligodeoxyribonucleotide. The oligonucleotide in the present invention may include naturally occurring dNMPs (i.e., dAMP, dGMP, dCMP and dTMP) or nucleotide analogs or derivatives. The oligonucleotide may also include a ribonucleotide. For example, the oligonucleotide in the present invention may include backbone-modified nucleotides, such as peptide nucleic acid (PNA) (M. Egholm et al., Nature, 365:566-568 (1993)), locked nucleic acid (LNA) (WO1999/014226), bridged nucleic acid (BNA) (WO2005/021570), phosphorothioate DNA, phosphorodithioate DNA, phosphoramidate DNA, amide-linked DNA, MMI-linked DNA, 2′-O-methyl RNA, alpha-DNA and methylphosphonate DNA, sugar-modified nucleotides, such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, and anhydrohexitol DNA, and base-modified nucleotides, such as C-5 substituted pyrimidine (the substituent including fluoro-, bromo-, chloro-, iodo-, methyl-, ethyl-, vinyl-, formyl-, ethynyl-, propynyl-, alkynyl-, thiazolyl-, imidazolyl-, and pyridyl-), 7-deazapurines with C-7 substituent (substituent including fluoro-, bromo-, chloro-, iodo-, methyl-, ethyl-, vinyl-, formyl-, alkynyl-, alkenyl-, thiazolyl-, imidazolyl-, and pyridyl-), inosine, and diaminopurine. Especially, as used herein, the term “oligonucleotide” is a single strand composed of deoxyribonucleotides. The term “oligonucleotide” includes oligonucleotides that hybridize with cleavage fragments occurring depending on a target nucleic acid sequence.

According to an embodiment of the present invention, the oligonucleotide is a probe and/or a primer.

As used herein, the term “primer” refers to an oligonucleotide that can act as a point of initiation of synthesis when placed under conditions in which synthesis of primer extension products complementary to a target nucleic acid strand (a template) is induced, i.e., in the presence of nucleotides and a polymerase, such as DNA polymerase, and appropriate temperature and pH conditions. The primer needs to be long enough to prime the synthesis of extension products in the presence of a polymerase. The appropriate length of the primer is determined according to a plurality of factors, including temperatures, fields of application, and primer sources.

As used herein, the term “probe” refers to a single-stranded nucleic acid molecule containing a portion or portions that are complementary to a target nucleic acid sequence. The probe may also contain a label capable of generating a signal for target detection.

The oligonucleotide may have typical primer and probe structure composed of a sequence hybridizing with a target nucleic acid sequence. Alternatively, the oligonucleotides may have distinctive structures through structural modification thereof. For example, the oligonucleotides may have structures of Scorpion primer, Molecular beacon probe, Sunrise primer, HyBeacon probe, tagging probe, DPO primer or probe (WO 2006/095981), and PTO probe (WO 2012/096523).

The oligonucleotide may be modified oligonucleotide, such as a degenerate base-containing oligonucleotide and/or a universal base-containing oligonucleotide, in which degenerate bases and/or universal bases are introduced into a conventional primer or probe. As used herein, the terms “conventional primer”, “conventional probe”, and “conventional oligonucleotide” refer to a typical primer, probe, and oligonucleotide, into which a degenerate base or non-natural base is not introduced. According to an embodiment of the present invention, in the degenerate base-containing oligonucleotide or universal base-containing oligonucleotide, at least 50%, at least 60%, at least 70%, at least 80%, at least 90%, or at least 95% are non-modified oligonucleotides. According to an embodiment of the present invention, the number of degenerate bases or universal bases introduced into the conventional oligonucleotide is in the range of specifically 7 or fewer, 5 or fewer, 4 or fewer, 3 or fewer, or 2 or fewer. Alternatively, the proportion of use of the degenerate bases and/or universal bases introduced into the conventional oligonucleotide is specifically 25% or less, 20% or less, 18% or less, 16% or less, 14% or less, 12% or less, 10% or less, 8% or less, or 6% or less. The proportion of use of the degenerate bases or universal bases represents a proportion of the degenerate bases or universal bases relative to a total of the nucleotides of the oligonucleotide into which the degenerate bases or universal bases are introduced. The degenerate bases include a variety of degenerate bases known in the art as follows: R: A or G; Y: C or T; S: G or C; W: A or T; K: G or T; M: A or C; B: C, G or T; D: A, G or T; H: A, C or T; V: A, C or G; N: A, C, G or T. The universal bases include a variety of universal bases known in the art as follows: deoxyinosine, inosine, 7-deaza-2′-deoxyinosine, 2-aza-2′-deoxyinosine, 2′-OMe inosine, 2′-F inosine, deoxy 3-nitropyrrole, 3-nitropyrrole, 2′-OMe 3-nitropyrrole, 2′-F3-nitropyrrole, 1-(2′-deoxy-beta-D-ribofuranosyl)-3-nitropyrrole, deoxy 5-nitropyrrole, 5-nitroindole, 2′-OMe 5-nitroindole, 2′-F5-nitroindole, deoxy 4-nitrobenzimidazole, 4-nitrobenzimidazole, deoxy 4-aminobenzimidazole, 4-aminobenzimidazole, deoxy nebularine, 2′-F nebularine, 2′-F4-nitrobenzimidazole, PNA-5-introindole, PNA-nebularine, PNA-inosine, PNA-4-nitrobenzimidazole, PNA-3-nitropyrrole, morpholino-5-nitroindole, morpholino-nebularine, morpholino-inosine, morpholino-4-nitrobenzimidazole, morpholino-3-nitropyrrole, phosphoramidate-5-nitroindole, phosphoramidate-nebularine, phosphoramidate-inosine, phosphoramidate-4-nitrobenzimidazole, phosphoramidate-3-nitropyrrole, 2′-O-methoxyethyl inosine, 2′-O-methoxyethyl nebularine, 2′-O-methoxyethyl 5-nitroindole, 2′-O-methoxyethyl 4-nitro-benzimidazole, 2′-O-methoxyethyl 3-nitropyrrole, and a combination thereof. More specifically, the universal base is deoxyinosine, inosine, or a combination thereof.

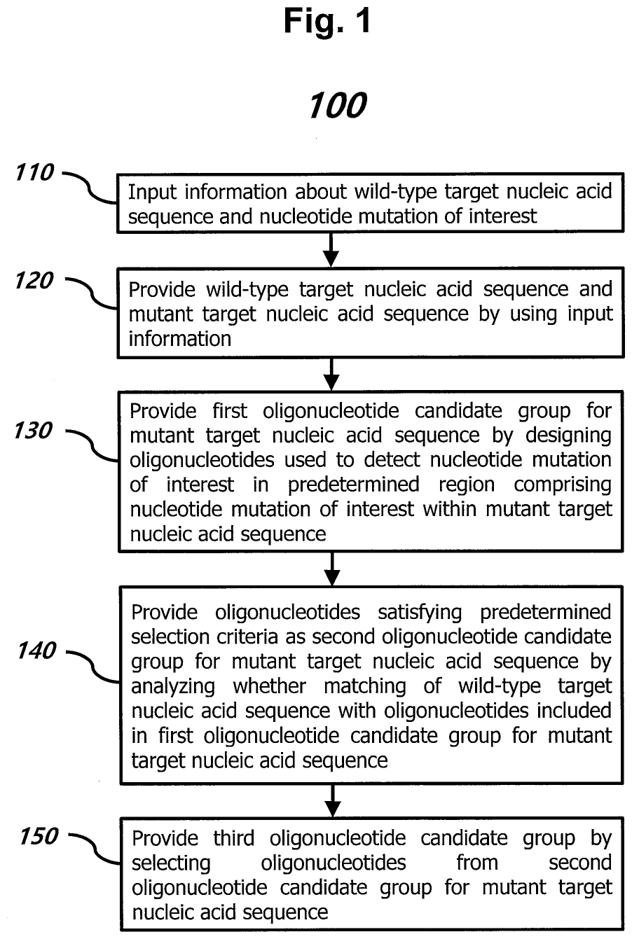

FIG. 1 is a flowchart of steps for performing a method of the present invention according to an embodiment of the present invention. The method of the present invention will be described in detail with reference to FIG. 1 as follows.

Step (a): Inputting information about wild-type target nucleic acid sequence and nucleotide mutation of interest (110)

First, the method of the present invention includes step (a) of inputting information about a wild-type target nucleic acid sequence and a nucleotide mutation of interest. The wild-type target nucleic acid sequence is a target nucleic acid sequence not comprising the nucleotide mutation of interest, the information includes information about (i) the wild-type target nucleic acid sequence, (ii) a position of the nucleotide mutation of interest occurring in the wild-type target nucleic acid sequence and (iii) wild-type and mutant bases at the position of the nucleotide mutation of interest, and wherein the position of the nucleotide mutation of interest is expressed as a start position and an end position.

The method of the present invention is directed to a computer-implemented method, and the inputting of the information about a wild-type target nucleic acid sequence and a nucleotide mutation of interest is performed through a user interface (UI).

In this specification, the step of inputting information represents the step of receiving the information. For example, the step of inputting information through the UI indicates the step of receiving the information through the UI.

The wild-type target nucleic acid sequence is a reference sequence for specifying the position of the nucleotide mutation occurring in the wild-type target nucleic acid sequence.

In the information, the wild-type target nucleic acid sequence may be obtained by identifying a gene having the nucleotide mutation of interest, retrieving a sequence from a publicly accessible database (specifically, NCBI nucleotide database) through the use of the gene, selecting organism information (e.g., taxonomy name or taxonomy ID) containing the nucleotide mutation of interest, and then retrieving the wild-type target nucleic acid from the Refseq database. Alternatively, the rs number information as an identifier for the mutation occurring in the wild-type target nucleic acid sequence may be identified and the wild-type target nucleic acid sequence may be retrieved from a publicly accessible database (specifically, NCBI's SNP database) through the rs number.

According to an embodiment of the present invention, the wild-type target nucleic acid sequence in step (a) has a predetermined length comprising a position of the nucleotide mutation of interest.

According to an embodiment of the present invention, the wild-type target nucleic acid sequence in step (a) has a predetermined length from a position of the nucleotide mutation of interest.

Specifically, the wild-type target nucleic acid sequence includes a sequence including a predetermined length upstream (left, −) and a predetermined length downstream (right, +) from the position of the nucleotide mutation of interest occurring in the wild-type target nucleic acid sequence. More specifically, the wild-type target nucleic acid sequence is a sequence including a predetermined length upstream (left, −) from the start position and a predetermined length downstream (right, +) from the end position of the nucleotide mutation of interest.

The predetermined length may be selected from, for example, 1500 bp to 100 bp, or may be 1500 bp, 1400 bp, 1300 bp, 1200 bp, 1100 bp, 1000 bp, 900 bp, 800 bp, 700 bp, 600 bp, 500 bp, 400 bp, 300 bp, 200 bp, or 100 bp.

As used herein while reciting directionality, the term “upstream” may be used interchangeably with “left” or “−” from any one position of a nucleic acid sequence, and the term “downstream” may be used interchangeably with “right” or “+” from any one position of a nucleic acid sequence.

As used herein while reciting the nucleotide length, the term “nucleotide” may be used interchangeably with “mer”, “base”, or “bp”.

In the information, the position of the nucleotide mutation of interest occurring in the wild-type target nucleic acid sequence is a position in the wild-type target nucleic acid sequence and is expressed as a start position and an end position in the wild-type target nucleic acid sequence. For example, when a substitution mutation of the T base with the G base occurs at position 305 in the wild-type target nucleic acid sequence, the start position and the end position of the mutation is position 305. When an inversion mutation of the CGTAT bases to the TATGC bases occurs at positions 333 to 337 in the wild-type target nucleic acid sequence, the start position of the mutation is position 333 and the end position thereof is position 337. When an insertion mutation of the TGC bases occurs between the G base at position 354 and the T base at position 355 in the wild-type target nucleic acid sequence, the start position and end position of the mutation are position 354. When a deletion mutation of the CAT bases occurs from the GCAT bases at positions 324 to 327 in the wild-type target nucleic acid sequence, the start position of the mutation is position 324 and the end position thereof is position 327. When a complex mutation of a deletion of the CAT bases and an insertion of the ACGACT occurs in the GCAT bases at positions 544 to 547 in the wild-type target nucleic acid sequence, the start position of the mutation is position 544 and the end position thereof is position 547. In cases where the length of the nucleic acid sequence is changed due to mutations, for example, in cases of an insertion mutation and a deletion mutation, a position before the position of each of insertion and deletion is expressed as a start position.

The information on wild-type and mutant bases at the positions of the nucleotide mutation of interest out of the information is as follows: for example, in the above description of the start position and the end position, the wild-type base is the T base and the mutant base is the G base for the substitution mutation, the wild-type bases are the CGTAT bases and the mutant bases are the TATGC bases for the inversion mutation, the wild-type base is the G base and the mutant bases are the GTGC bases for the insertion mutation, the wild-type bases are the GCAT bases and the mutant base is the G base for the deletion mutation, and the wild-type bases are the GCAT bases and the mutant bases are the GACGACT bases for the complex mutation. In cases where the length of the nucleic acid sequence is changed due to a mutation, for example, in cases of an insertion mutation and a deletion mutation, information also including a base before the position of each of insertion and deletion may be input.

Step (b): Providing wild-type target nucleic acid sequence and mutant target nucleic acid sequence (120)

Next, the method of the present invention includes step (b) of providing the wild-type target nucleic acid sequence and a mutant target nucleic acid sequence by using the input information. The mutant target nucleic acid sequence is a target nucleic acid sequence comprising the nucleotide mutation of interest, and the wild-type and mutant target nucleic acid sequences include forward and reverse wild-type target nucleic acid sequences, and forward and reverse mutant target nucleic acid sequences, respectively.

In the present step, templates as object for designing oligonucleotides in the step to be described later are provided.

The wild-type and mutant target nucleic acid sequences are provided using the information input in step (a). Specifically, the mutant target nucleic acid sequence may be provided by applying the position information and mutant base information of the nucleotide mutation of interest occurring in the wild-type target nucleic acid sequence in step (a) to the wild-type target nucleic acid sequence. In addition, a reverse wild-type target nucleic acid sequence is provided if the wild-type target nucleic acid sequence is a forward wild-type target nucleic acid sequence, and a reverse mutant target nucleic acid sequence may be provided from the forward mutant target nucleic acid sequence if the provided mutant target nucleic acid sequence is provided from the forward wild-type target nucleic acid sequence.

In other words, when the information about a forward wild-type target nucleic acid sequence is input in step (a), a forward (5′ to 3′) wild-type target nucleic acid sequence and a reverse complementary (3′ to 5′) wild-type target nucleic acid sequence are provided as templates. Through the information, input in step (a), about the position of the nucleotide mutation of interest occurring in the wild-type target nucleic acid sequence, and wild-type and mutant bases at the position of the nucleotide mutation of interest, a forward (5 ‘to 3’) mutant target nucleic acid sequence and a reverse complementary (3′ to 5′) mutant target nucleic acid sequence are provided as templates.

As used herein while reciting the directivity of the target nucleic acid sequence as a template, the term “forward” refers to the directivity from the 5′ end to the 3′ end, and the term “reverse” refers to the directivity from the 3′ end to the 5′ end, which is complementary to the forward target nucleic acid sequence.

As used herein while reciting the wild-type and mutant target nucleic acid sequences, the term “providing” refers to the providing of sequence information of the target nucleic acid sequence.

According to an embodiment of the present invention, the mutant target nucleic acid sequence comprises information about the nucleotide mutation of interest as well as other nucleotide mutations. For example, when there is a mutation having a non-conservative base at any position of multiple target nucleic acid sequences collected besides a nucleotide mutation of interest to be detected, information about such a mutation may also be included in the mutant target nucleic acid sequence. The mutations having the non-conservative base may be different or same according to the organism or continent.

As used herein, the term “non-conservative” refers to not showing conservativeness at alignment positions of multiple target nucleic acid sequences, the term means that at alignment positions of multiple target nucleic acid sequences, the proportion or number of a different particular type of bases to all the bases exceeds a predetermined value. For example, as for another nucleotide mutation besides the nucleotide mutation of interest, the proportion of different particular type of bases to all the bases is at least 0.1%, at least 0.2%, at least 0.3%, at least 0.4%, at least 0.5%, at least 0.6%, or at least 0.7%, but is not limited thereto.

Step (c): Providing first oligonucleotide candidate group for mutant target nucleic acid sequence (130)

The method of the present invention includes step (c) of providing a first oligonucleotide candidate group for the mutant target nucleic acid sequence by designing oligonucleotides used to detect the nucleotide mutation of interest in a predetermined region comprising the nucleotide mutation of interest within the mutant target nucleic acid sequence.

In the present step, the first oligonucleotide candidate group for the mutant target nucleic acid sequence are provided by designing oligonucleotides used to detect the mutant target nucleic acid sequence.

Since the position of the nucleotide mutation of interest occurring within the wild-type nucleic acid sequence and the mutant base at the position are known in step (a), the oligonucleotide designing in the present step is performed in a predetermined region containing the nucleotide mutation of interest within the mutant target nucleic acid sequence. The predetermined region comprising the nucleotide mutation of interest may be designated as an oligonucleotide design range.

According to an embodiment of the present invention, the oligonucleotides in step (c) are designed to have matching or complementary sequences to the predetermined region comprising the nucleotide mutation of interest within the mutant target nucleic acid sequence.

According to the present embodiment, the oligonucleotides to be designed are designed to have matching or complementary sequences to the oligonucleotide design range.

The predetermined region comprising the nucleotide mutation of interest, as the oligonucleotide design range, may be selected from 20 to 200 bp, 30 to 180 bp, 40 to 160 bp, 50 to 150 bp, 60 to 130 bp, or 70 to 120 bp.

The predetermined region comprising the nucleotide mutation of interest, as the oligonucleotide design range, is a region including a predetermined length upstream and a predetermined length downstream from the position of the nucleotide mutation of interest present within the mutant target nucleic acid sequence, and the predetermined length upstream may be selected from 0 bp to 60 bp and the predetermined length downstream may be selected from 0 bp to 60 bp. The predetermined length upstream and the predetermined length downstream may be same or different. For example, if the oligonucleotide design range is a region including 0 bp upstream and 60 bp downstream from the position of the nucleotide mutation of interest, this means that oligonucleotides are designed in a region including 60 bp downstream from the nucleotide mutation of interest within the mutant target nucleic acid sequence. Alternatively, if the oligonucleotide design range is a region including 10 bp upstream from the start position of the nucleotide mutation of interest and 60 bp downstream from the end position thereof with 10 bp in length between the start position and the end position, this means that oligonucleotides are designed in a region of 80 bp within the mutant target nucleic acid sequence.

According to an embodiment of the present invention, the oligonucleotides in step (c) are designed according to lengths at positions from a predetermined position upstream of the start position of the nucleotide mutation of interest within the mutant target nucleic acid sequence to the end position thereof. Particularly, the oligonucleotides in step (c) are designed according to predetermined lengths at positions from a predetermined position upstream of the start position of the nucleotide mutation of interest within the mutant target nucleic acid sequence to the end position thereof. Particularly, the oligonucleotides in step (c) are designed according to predetermined lengths at each position from a predetermined position upstream of the start position of the nucleotide mutation of interest within the mutant target nucleic acid sequence to the end position thereof. Alternatively, the oligonucleotides in step (c) are designed according to lengths at each position from a predetermined position upstream of the start position of the nucleotide mutation of interest within the mutant target nucleic acid sequence to the end position thereof.

The position of the nucleotide mutation of interest present within the mutant target nucleic acid sequence may represent the position of the nucleotide mutation of interest present within the mutant target nucleic acid sequence provided in step (b), and the position of the mutation may be expressed as a start position and an end position.

The predetermined position upstream of the start position of the nucleotide mutation of interest may be selected from 1 to 50 bp or 2 to 40 bp, and for example may be 1 bp, 2 bp, 3 bp, 4 bp, 38 bp, 39 bp, 40 bp, or 41 bp.

The lengths of the oligonucleotides to be designed according to lengths (particularly, predetermined lengths) may be, for example, 10-60 nucleotides, 10-50 nucleotides, 10-45 nucleotides, 10-40 nucleotides, 10-35 nucleotides, 15-60 nucleotides, 15-50 nucleotides, 15-45 nucleotides, 15-40 nucleotides, 15-35 nucleotides, 20-60 nucleotides, 20-50 nucleotides, 20-45 nucleotides, 20-40 nucleotides, or 20-35 nucleotides.

In designing the oligonucleotide according to the present embodiment, for example, when the predetermined position upstream of the start position of the nucleotide mutation of interest is 2 bp, the lengths of oligonucleotides to be designed according to lengths (predetermined lengths) are 3 to 4 nucleotides, the mutant target nucleic acid sequence is 5′-ATGCGGTTG-3′, and the C base at the fourth position of the 5′-end of the mutant target nucleic acid sequence is a mutant base, which corresponds to a substitution mutation of the A base into the C base, oligonucleotides are designed according to the 3- to 4-nucleotide lengths at each position from the T base to the C base, which are 2 bp upstream from the C base, which corresponds to the start position and end position of the mutation within the mutant target nucleic acid sequence (i.e., 5′-TGC-3′, 5′-TGCG-3′, 5′-GCG-3′, 5′-GCGG-3′, 5′-CGG-3′, and 5′-CGGT-3′).

As used herein, the phrase “oligonucleotides having the same start position for designing an oligonucleotide” refer to oligonucleotides with the same start position for designing oligonucleotides according to lengths (by length). In the above example, the oligonucleotides 5′-TGC-3′ and 5′-TGCG-3′ are oligonucleotides having the same start position for designing an oligonucleotide.

In designing the oligonucleotide according to the present embodiment, for example, when the predetermined position upstream from the start position of the nucleotide mutation of interest is 2 bp, the lengths of oligonucleotides to be designed according to the lengths (by length) are 3 to nucleotides, the mutant target nucleic acid sequence is 5′-ATGCTCTGGC-3′, and there is an insertion mutation of CT, which are bases at the fourth and fifth positions from the A base as the 5′-end of the mutant target nucleic acid sequence, oligonucleotides may be designed according to the 3- to 5-nucleotide lengths at each position from the T base, which is present 2 bp upstream from the C base present at the start position of the nucleotide mutation of interest, to the T base, which is present at the end position of the nucleotide mutation of interest. Specifically, 5′-TGC-3′, 5′-TGCT-3, 5′-TGCTC-3′, 5′-GCT-3, 5′-GCTC-3′, 5′-GCTCT-3, 5′-CTC-3′, 5′-CTCT-3, 5′-CTCTG-3′, 5′-TCT-3, 5′-TCTG-3′, and 5′-TCTGG-3′ are designed from T, which is a second base from the 5′-end of the mutant target nucleic acid sequence, that is, T, which is present 2 bp upstream from the C base present at the start position of the mutation. Also, oligonucleotides are designed in the same manner for reverse sequences of the mutant target nucleic acid sequence.

In the above-described example for oligonucleotide designing with respect to the insertion mutation, the A base at the 5′-end is designated as the first position when the position from the 5′-end of the mutant target nucleic acid sequence is described, and the G base upstream of C, which is a base at the start position of the nucleotide mutation of interest, is designated as the first position when a predetermined position upstream from the start position of the nucleotide mutation of interest is described.

In other words, when a reference point is based on an end of a nucleic acid sequence, the base present at the end of the nucleic acid sequence is included and the base is considered as the first base, and when a reference point is based on a base present within a nucleic acid sequence, a base upstream or downstream of the base, excluding the base, is considered as the first base. When a reference point is based on a base present within a nucleic acid sequence, a different description from the above will be separately made.

According to an embodiment of the present invention, the oligonucleotides to be designed in step (c) are designed to satisfy at least one of the following conditions:
(i) a length of 10-60 nucleotides;
(ii) a Tm value of 50-85° C.;
(iii) exclusion of a G-run sequence with at least three Gs; and
(iv) a GC content of 40% or more in the 5′-end portion.

The oligonucleotide design conditions include more specifically at least two, still more specifically at least three, and still more specifically at least four of the aforementioned conditions.

The length among the design conditions is, for example, 10-60 nucleotides, 10-50 nucleotides, 10-45 nucleotides, 10-40 nucleotides, 10-35 nucleotides, 15-60 nucleotides, 15-50 nucleotides, 15-45 nucleotides, 15-40 nucleotides, or 15-35 nucleotides.

The Tm value among the design conditions is, for example, 50-80° C., 50-75° C., 55-80° C., 55-75° C., 60-80° C., 60-75° C., 65-80° C., or 60-75° C. Specifically, the Tm value among the design conditions is 55-80° C., 60-78° C., 63-78° C., 65-75° C., 67-75° C. or 65-73° C.

The G-run sequence among the design conditions is directed to the exclusion of a G-run sequence with at least three Gs or at least four Gs.

The GC content at the 5′-end portion of the oligonucleotides is 40% or more, specifically, 40-70% or 40-60%. The 5′-end portion means a portion within 10 nucleotides from the 5′-end of the oligonucleotides.

Step (d): Providing oligonucleotides satisfying selection criteria as second oligonucleotide candidate group by analyzing matching of wild-type target nucleic acid sequence with first oligonucleotide candidate group (140)

Then, the method of the present invention includes step (d) of providing oligonucleotides satisfying the following selection criteria as a second oligonucleotide candidate group for the mutant target nucleic acid sequence by analyzing matching of the wild-type target nucleic acid sequence with the oligonucleotides included in the first oligonucleotide candidate group for the mutant target nucleic acid sequence. The selection criteria include that (i) the number of mismatches between the wild-type target nucleic acid sequence and a predetermined region at the 5′-end, middle or 3′-end of an oligonucleotide is one or more; or (ii) the ratio of mismatches between the wild-type target nucleic acid sequence and an oligonucleotide is a predetermined value or more.

In the present step, oligonucleotides satisfying a predetermined selection criteria are selected so as to allow the oligonucleotides designed for the mutant target nucleic acid sequence to distinguishably detect the mutant target nucleic acid sequence from the wild-type target nucleic acid sequence. Such a selection procedure can solve the problem of the conventional art in that different methods of designing oligonucleotides need to be performed according to the type of nucleotide mutation.

A region of the wild-type target nucleic acid sequence to be analyzed for matching with the oligonucleotides included in the first oligonucleotide candidate group may be all or a part of the wild-type target nucleic acid sequence. Specifically, the position of the nucleotide mutation of interest occurring within the wild-type nucleic acid sequence is known, and thus a matching analysis may be performed on a partial region of the wild-type target nucleic acid sequence, which corresponds to a sequence region of the mutant target nucleic acid sequence in which oligonucleotides used to detect the nucleotide mutation of interest are designed.

The matching analysis may be performed by various methods known in the art. For example, a matching analysis may be performed while an oligonucleotide designed for the mutant target nucleic acid sequence is moved to a sequence region of the wild-type target nucleic acid sequence, corresponding to the oligonucleotide design range, or a matching analysis may be performed by performing BLAST on the wild-type target nucleic acid sequence using the designed oligonucleotide as a query sequence.

The matching analysis may be performed on between the target nucleic acid sequence and the oligonucleotide having the same directionality.

The selection criteria for allowing the method of the present invention to design oligonucleotides through the integration of the types of nucleotide mutations include the following: (i) the number of mismatches between the wild-type target nucleic acid sequence and a predetermined region at the 5′-end, middle or 3′-end of the oligonucleotide is one or more; or (ii) the ratio of mismatches between the wild-type target nucleic acid sequence and the oligonucleotide is a predetermined value or more.

The selection criterion (i) is a criterion capable of distinguishably detecting between the mutant target nucleic acid sequence and the wild-type target nucleic acid sequence due to the mismatching between a certain partial region of the oligonucleotide and the wild-type target nucleic acid sequence. Here, the region other than the certain partial region of the oligonucleotide comprises a sequence matched with the wild-type target nucleic acid sequence.

The certain partial region of the oligonucleotide for such a distinguishable detection is a predetermined region of the 5′-end, the middle, or the 3′-end of the oligonucleotide, specifically, a predetermined region of the 5′-end or the middle of the oligonucleotide, or a predetermined region of the 5′-end or 3′-end of the oligonucleotide, more specifically a predetermined region of the 5′-end of the oligonucleotide. The predetermined region may be, for example, a region within 10 nucleotides, 7 nucleotides, 5 nucleotides, 3 nucleotides, or 2 nucleotides or a region of 1 nucleotide from the 5′-end or the 3′-end of the oligonucleotide. The predetermined region of the middle of the oligonucleotide may be a region within 5 nucleotides, 3 nucleotides, or 2 nucleotides upstream and downstream from a nucleotide of the middle including the nucleotide of the middle, or the nucleotide of the middle.

Examples in which a mutant target nucleic acid sequence can be distinguishably detected from a wild-type target nucleic acid sequence due to the mismatching between the wild-type target nucleic acid sequence and a predetermined region of the 5′-end, middle, or 3′-end of an oligonucleotide are as follows:

In a case where an oligonucleotide is designed to comprise a base corresponding to a nucleotide mutation of interest, a target nucleic acid sequence complementary to a mutant target nucleic acid sequence comprising the nucleotide mutation of interest is described as a matching template herein and a target nucleic acid sequence complementary to a wild-type target nucleic acid sequence not comprising the nucleotide mutation of interest is described as a mismatching template herein.

For example, assuming that an oligonucleotide satisfying the aforementioned selection criterion (i) is used in the PTOCE method (WO 2012/096523) or the VD-PTOCE method (WO 2013/133561) developed by the present applicant, and when a predetermined region of the 5′-end (e.g., 5′-end) of the 3′-targeting portion (the aforementioned oligonucleotide designed in the present invention) of the probing and tagging oligonucleotide (PTO) has a complementary base to a matching template and a non-complementary base to a mismatching template, for example, under upstream primer-dependent cleavage induction, the cleavage of the PTO hybridized with the matching template may be induced at a position immediately adjacent in a 3′-direction to the 5′-end of the 3′-targeting portion of the PTO. The PTO fragment is hybridized with a capturing and templating oligonucleotide (CTO) having a capturing portion including a sequence corresponding to the nucleotide mutation of interest and then extended to form the extended duplex, providing a target signal.

If the same PTO is hybridized with the mismatching template having the identical sequence to the matching template except for the nucleotide mutation of interest, the cleavage of the PTO may occur at a position two nucleotides apart in a 3′-direction from the 5′-end of the 3′-targeting portion of the PTO. The 3′-end of the PTO fragment has the nucleotide being cleaved additionally in addition to the complementary nucleotide to the single nucleotide variation. In a case where the site of the CTO hybridized with the additionally cleaved nucleotide is designed to have a non-complementary sequence to the nucleotide being cleaved additionally, the 3′-end of the PTO fragment is not hybridized with the CTO, resulting in no extension of the PTO fragment in a controlled condition. Even if the PTO fragment is extended to form the extended duplex, the duplex has a different Tm value from the duplex derived from hybridization between the PTO and the mismatching template.

Through such a manner, the oligonucleotide satisfying the aforementioned selection criterion (i) can distinguishably detect the mutant target nucleic acid sequence from the wild-type nucleic acid sequence.

In addition, an oligonucleotide satisfying a criterion associated with mismatching with a predetermined region of the 3′-end of the oligonucleotide in the selection criterion (i) can distinguishably detect a mutant target nucleic acid sequence by the following method.

For example, in a case where a mismatch exists in the 3′-end of an upstream primer, the 3′-end of the upstream primer is annealed to the matching template and extended to induce the PTO cleavage. The PTO fragment as a product is hybridized with CTO to provide a target signal. In contrast, in a case where the 3′-end of the upstream primer mismatches a nucleotide mutation in a mismatching template, extension does not occur under conditions that annealing of the 3′-end of the primer is essential for extension even when the upstream primer is hybridized with the mismatching template, and thus a target signal is not generated.

In addition, an oligonucleotide satisfying a criterion associated with mismatching in a predetermined region of the middle of the oligonucleotide in the selection criterion (i) can distinguishably detect a mutant target nucleic acid sequence by the following method.

For example, in a case where a sequence for the nucleotide mutation of interest is located in the middle of the 3′-targeting portion (the aforementioned oligonucleotide designed in the present invention) of PTO, the PTO is hybridized with the matching template under controlled conditions and then cleaved. The PTO fragment as a product is hybridized with the CTO to provide a target signal. On the other hand, under controlled conditions, the PTO is not hybridized with the mismatching template and is not cleaved.

According to an embodiment of the present invention, the selection criterion (i) is that the number of mismatches between the wild-type target nucleic add sequence and a predetermined region at the 5′-end of the oligonucleotide is one or more, and most specifically, the number of mismatches between the wild-type target nucleic acid sequence and the 5′-end of the oligonucleotide is one.

According to an embodiment of the present invention, the mismatches (i) in step (d) are minimum mismatches between the wild-type target nucleic acid sequence and the predetermined region of the 5′-end, middle, or 3′-end of the oligonucleotide.

For example, in a case where the predetermined region of the 5′-end of the oligonucleotide in the selection criterion (i) is a 2-mer, the designed oligonucleotide is 5′-ATGCT-3′, and the region of the wild-type target nucleic acid sequence to be subjected to a matching analysis is 5′-AAGCT-3′, one minimum mismatch exists when a matching analysis is performed on 5′-AAGCT-3′, which is an object of matching analysis, with respect to AT, which are 2-mer bases of the 5′-end of the oligonucleotide, and thus the designed oligonucleotide 5′-AATGC-3′ is selected and provided as a second oligonucleotide candidate group.

The selection criterion (ii) is a criterion capable of distinguishably detecting the mutant target nucleic acid sequence from the wild-type target nucleic acid sequence in a manner in which the oligonucleotide is hybridized with the mutant target nucleic acid sequence (or sequence complementary to the mutant target nucleic acid sequence) and is non-hybridized with the wild-type target nucleic acid sequence (or sequence complementary to the wild-type target nucleic acid sequence) due to a predetermined value of the ratio of mismatches between the entire sequence of the oligonucleotide and the wild-type target nucleic acid sequence.

For example, assuming that an oligonucleotide satisfying the aforementioned selection criterion (ii) is used in the PTOCE method (WO 2012/096523) developed by the present applicant, and when the 3′-targeting portion (the aforementioned oligonucleotide designed in the present invention) of the probing and tagging oligonucleotide (PTO) has a complementary base to the mutant target nucleic acid sequence and at least a predetermined proportion of non-complementary bases so as to non-hybridize with the wild-target nucleic acid sequence, for example, under upstream primer-dependent cleavage induction, the cleavage of the PTO hybridized with the mutant target nucleic acid sequence may be induced at any one position of the 5′-end of the 3′-targeting portion of the PTO. The PTO fragment is hybridized with a capturing and templating oligonucleotide (CTO) having a capturing portion including a sequence complementary to the fragment and then extended to form the extended duplex, thereby providing a target signal.

If the same PTO contains at least a predetermined proportion of non-complementary bases to the wild-type target nucleic acid sequence including the nucleotide mutation of interest, the PTO is not hybridized to the wild-type target nucleic acid sequence and thus is not cleaved, and the PTO is not extended even if hybridizing with CTO.

Through such a manner, the oligonucleotide satisfying the aforementioned selection criterion (ii) can distinguishably detect the mutant target nucleic acid sequence from the wild-type nucleic acid sequence.

The predetermined value of the ratio of mismatches may be selected such that the oligonucleotide designed for the mutant target nucleic acid sequence is not hybridized with the wild-type target nucleic acid sequence. For example, the predetermined value of the ratio of mismatches may be selected from 10% to 100%, or may be 10%, 20%, 30%, 40%, 50%, 60%, 70%, 80%, 90%, or 100%.

According to an embodiment of the present invention, the mismatches (ii) in step (d) indicate minimum mismatches between the wild-type target nucleic acid sequence and the oligonucleotide. More particularly, the mismatches (ii) in step (d) indicate the number of minimum mismatches between the wild-type target nucleic acid sequence and the oligonucleotide

In the present embodiment, at least a predetermined value of the ratio of mismatches relative to the sequence of the oligonucleotide indicates the ratio of the number of mismatches in an oligonucleotide showing the minimum mismatches between the wild-type target nucleic acid sequence and the oligonucleotide as a result of the matching analysis between the wild-type target nucleic acid sequence and the entire sequence of the oligonucleotide.

For example, in a case where the number of mismatches is 20 mer, 17 mer, 10 mer, 18 mer, or 21 mer as a result of the matching analysis of an oligonucleotide designed to have 30 mer with respect to the wild-type target nucleic acid sequence, the number of minimum mismatches out of these values is 10 mer, and the sequence of the oligonucleotide is a 30-mer, and thus the ratio of mismatches is 33.3%. If the predetermined value of the ratio is 30% in the above case, the designed oligonucleotide satisfies the selection criterion (ii) and thus is provided as a second oligonucleotide candidate group.

According to an embodiment of the present invention, the selection criteria include the following: (i) the number of mismatches between the wild-type target nucleic acid sequence and a predetermined region at the 5′-end, middle or 3′-end of an oligonucleotide is one or more; and (ii) the ratio of mismatches between the wild-type target nucleic acid sequence and an oligonucleotide is a predetermined value or more.

According to an embodiment of the present invention, the second oligonucleotide candidate group includes a modified oligonucleotide having an increased target-coverage by substituting at least one base of the designed oligonucleotide with a degenerate base and/or a universal base so as to increase the target-coverage for the mutant target nucleic acid sequence containing other nucleotide mutations as well as the nucleotide mutation of interest.

According to an embodiment of the present invention, the method further includes, after step (d), step d-1) of arranging the oligonucleotides included in the second oligonucleotide candidate group by giving ranks according to at least one criterion of the following arrangement criteria:
(i) the position of a mismatch in an oligonucleotide as a result of the matching analysis in step (d); wherein the closer the position of the mismatch is to the 5′-end, 3′-end, or middle, the higher the rank,
(ii) the number of mismatches of an oligonucleotide as a result of the matching analysis in step (d); wherein the larger the number of the mismatches, the higher the rank,
(iii) a Tm value of an oligonucleotide; wherein the higher the Tm value, the higher the rank,
(iv) a GC content in a predetermined region of the 5′-end, middle, or 3′-end of an oligonucleotide; wherein the more the GC content, the higher the rank,
(v) the number of consecutive G bases included in an oligonucleotide; wherein the smaller the number of consecutive G bases, the higher the rank,
(vi) the number or proportion of consecutive nucleotides involved in the formation of a homodimer when the oligonucleotide forms the homodimer; wherein the smaller the number or proportion, the higher the rank,
(vii) a hairpin structure-forming free energy value (ΔG value); wherein the larger the free energy value, the higher the rank, and
(viii) a length; wherein the shorter the length, the higher the rank.

In the present embodiment, ranks are given for providing a third oligonucleotide candidate group by selecting the second oligonucleotide candidate group in step (e) to be described later.

According to the present embodiment, the oligonucleotides included in the second oligonucleotide candidate group are arranged to satisfy at least one (specifically, arrangement criterion (i)), specifically at least two, more specifically at least three, at least four, at least five, at least six, at least seven, or at least eight, and most specifically eight of the arrangement criteria considering ranks.

There may be largely two manners in the arrangement of the oligonucleotides included in the second oligonucleotide candidate group:

According to a first manner, the at least two arrangement criteria are different in criticality, and the oligonucleotides may be arranged to satisfy the arrangement criterion with the highest criticality (e.g., arrangement criterion (i)).

If multiple oligonucleotides satisfy the arrangement criterion with the highest criticality, the oligonucleotides may be arranged to satisfy the next-order arrangement criterion.

For example, if the criticality of the arrangement criteria is the order of arrangement criteria (i), (ii), (iii), (iv), (v), (vi), (vii) and (viii), and three oligonucleotides satisfy arrangement criterion (i), the three oligonucleotides are arranged according to arrangement criterion (ii). If three oligonucleotides satisfy arrangement criterion (ii), the oligonucleotides are arranged to satisfy arrangement criterion (iii).

According to a second manner, different weights are assigned to the arrangement criteria and scores are assigned to values (or value ranges) in each of the arrangement criteria, and thus, the total score of each of the oligonucleotides may be obtained considering ranks, and considering the calculated total scores, the oligonucleotides may be arranged.

According to an embodiment of the present invention, the at least two arrangement criteria are different in criticality, and the method of the present invention further includes a step of arranging the oligonucleotides included in the second oligonucleotide candidate group to satisfy the at least two arrangement criteria considering the criticality.

Step (e): Providing third oligonucleotide candidate group (150)

Last, the method of the present invention includes step (e) of providing a third oligonucleotide candidate group by selecting oligonucleotides from the second oligonucleotide candidate group for the mutant target nucleic acid sequence. The third oligonucleotide candidate group is used to detect the nucleotide mutation of interest in the target nucleic acid sequence.

According to an embodiment of the present invention, the third oligonucleotide candidate group in step (e) is selected by a method including the following steps:
(e-1) arranging by giving ranks according to at least one of the following arrangement criteria to oligonucleotides having the same start position for designing an oligonucleotide among the oligonucleotides included in the second oligonucleotide candidate group; and
(e-2) selecting the highest ranked oligonucleotide from oligonucleotides having the same start position for designing the arranged oligonucleotides, and wherein the arrangement criteria include the following:
(i) the position of a mismatch in an oligonucleotide as a result of the matching analysis in step (d); wherein the closer the position of the mismatch is to the 5′-end, 3′-end, or middle, the higher the rank,
(ii) the number of mismatches in an oligonucleotide as a result of the matching analysis in step (d); wherein the larger the number of the mismatches, the higher the rank,
(iii) a Tm value of an oligonucleotide; wherein the higher the Tm value, the higher the rank,
(iv) a GC content in a predetermined region of the 5′-end, middle, or 3′-end of an oligonucleotide; wherein the higher the GC content, the higher the rank,
(v) the number of consecutive G bases included in an oligonucleotide; wherein the smaller the number of consecutive G bases, the higher the rank,
(vi) the number or proportion of consecutive nucleotides involved in the formation of a homodimer when the oligonucleotide forms the homodimer; wherein the smaller the number or proportion, the higher the rank,
(vii) a hairpin structure-forming free energy value (OG value); wherein the larger the free energy value, the higher the rank, and
(viii) a length; wherein the shorter the length, the higher the rank.

In the present embodiment, oligonucleotides having the same start position for designing an oligonucleotide, among the oligonucleotides included in the second oligonucleotide candidate group, are arranged by giving ranks, and then the highest ranked oligonucleotide is selected. Since the oligonucleotides having the same start position for designing an oligonucleotide, among the oligonucleotides included in the second oligonucleotide candidate group, would be present according to the oligonucleotide design position in the mutant target nucleic acid sequence, the highest ranked oligonucleotide is selected according to each design position.

The present embodiment is different from the step d-1) in that in step d-1), the oligonucleotide objects to be given ranks and arranged are all the oligonucleotides included in the second oligonucleotide candidate group, but in the present embodiment, the oligonucleotide objects to be given ranks and arranged are oligonucleotides having the same start position for designing an oligonucleotide among the oligonucleotides included in the second oligonucleotide candidate group.

According to the present embodiment, in step (e-1), the oligonucleotides having the same start position for designing an oligonucleotide among the oligonucleotides included in the second oligonucleotide candidate group are arranged to satisfy at least one (specifically, arrangement criterion (i)), specifically at least two, more specifically at least three, at least four, at least five, or at least six, at least seven, or at least eight, and most specifically eight of the arrangement criteria considering ranks.

There may be largely two manners in the arrangement of the oligonucleotides having the same start position for designing an oligonucleotide among the oligonucleotides included in the second oligonucleotide candidate group:

According to a first manner, the at least two arrangement criteria are different in criticality, and the oligonucleotides may be arranged to satisfy the arrangement criterion with the highest criticality (e.g., arrangement criterion OD.

If multiple oligonucleotides satisfy the arrangement criterion with the highest criticality, the oligonucleotides may be arranged to satisfy the next-order arrangement criterion.

For example, if the criticality of the arrangement criteria is the order of arrangement criteria (i), (ii), (iii), (iv), (v), (vi), (vii) and (viii), and three oligonucleotides satisfy arrangement criterion (i), the three oligonucleotides are arranged according to arrangement criterion (ii). If three oligonucleotides satisfy arrangement criterion (ii), the oligonucleotides may be arranged to satisfy arrangement criterion (iii) to select the highest ranked oligonucleotides as a third oligonucleotide candidate group.

According to a second manner, different weights are assigned to the arrangement criteria and scores are assigned to values (or value ranges) in each of the arrangement criteria, and thus, the total score of each of the oligonucleotides may be obtained considering ranks, and considering the calculated total scores, the oligonucleotides may be arranged, and the highest-ranked oligonucleotides may be selected as the third oligonucleotide candidate group through the ranks according to total scores.

According to an embodiment of the present invention, the at least two arrangement criteria are different in criticality, and the method of the present invention further includes a step of arranging the oligonucleotides having the same start position for designing an oligonucleotide among the oligonucleotides included in the second oligonucleotide candidate group to satisfy the at least two arrangement criteria considering the criticality.

Through the aforementioned method, the third oligonucleotide candidate group used to detect a nucleotide mutation of interest in the target nucleic acid sequence may be provided.

According to an embodiment of the present invention, the first to third oligonucleotide candidate groups are first to third probe candidate groups, respectively and the method further includes, after step (e), the following steps:
(f) providing a primer candidate group for the mutant target nucleic acid sequence by designing primers to amplify a predetermined region comprising the nucleotide mutation of interest within the mutant target nucleic acid sequence; and
(g) providing a combination of a probe and primers by combining the third probe candidate group and the primer candidate group.

The present embodiment shows a procedure wherein the first to third probe candidate groups are provided, the primer candidate group is provided, and then a combination of a probe and primers is provided by combining the third probe candidate group and the primer candidate group.

Step (f): Providing primer candidate group for mutant target nucleic acid sequence

In step (f), primers are designed to amplify a predetermined region containing the nucleotide mutation of interest within the mutant target nucleic acid sequence.

A primer design range, that is, the predetermined range comprising the nucleotide mutation of interest to be amplified using a primer is different from a region (i.e., a probe design range) for designing a probe included in the first probe candidate group in step (c), but the primer design range may be set to amplify a region including the predetermined region for designing the probe.

The primer design range, that is, the predetermined range comprising the nucleotide mutation of interest to be amplified using a primer may be selected within a region of 500 bp (specifically, 400 bp, 350 bp, 300 bp, 250 bp, 200 bp, 150 bp, 100 bp, or 50 bp) upstream of the start position of the nucleotide mutation of interest to 500 bp (specifically, 400 bp, 350 bp, 300 bp, 250 bp, 200 bp, 150 bp, 100 bp, or 50 bp) downstream of the end position of the nucleotide mutation of interest.

According to an embodiment of the present invention, the primers to be designed in step (f) are designed to satisfy at least one of the following conditions:
(i) a Tm value of 40-70° C.;
(ii) a length of 15-50 nucleotides; and
(iii) exclusion of a G-run sequence with at least five Gs.

The Tm value among the design conditions is for example 40-70° C., 50-70° C., 55-70° C., 45-65° C., 50-65° C., 55-65° C., 45-60° C., or 50-65° C. Specifically, the Tm value among the design conditions is for example 40-70° C., 45-65° C., 50-65° C., 50-60° C., 55-65° C., or 55-60° C.

The length among the design conditions is, for example, 15-60 nucleotides, 15-50 nucleotides, 15-45 nucleotides, 15-40 nucleotides, 15-35 nucleotides, 15-30 nucleotides, 15-25 nucleotides, 18-45 nucleotides, 18-40 nucleotides, 18-35 nucleotides, 18-30 nucleotides, or 18-25 nucleotides. Specifically, the length among the design conditions is, for example, 15-40 nucleotides, 16-40 nucleotides, 17-40 nucleotides, 18-40 nucleotides, 15-35 nucleotides, 16-35 nucleotides, 17-35 nucleotides, 18-35 nucleotides, 15-30 nucleotides, 16-30 nucleotides, 17-30 nucleotides, 18-30 nucleotides, 18-25 nucleotides, or 17-25 nucleotides.

The G-run sequence among the design conditions is the exclusion of a G-run sequence with, for example, at least five Gs.

In cases where the primer is a DPO primer developed by the present applicant (see U.S. Pat. No. 8,092,997), the descriptions for the Tm and the length of the DPO primer disclosed in the patent document may be provided as the design conditions.

The primer design conditions include more specifically at least two, and still more specifically at least three of the above-described conditions.

According to an embodiment of the present invention, the method further includes, after step (f), the following steps:
(f-1) arranging by giving ranks according to at least one of the following arrangement criteria to primers having the same start position for designing a primer among the primers included in the primer candidate group; and
(f-2) selecting the highest ranked primer from primers having the same start position for designing the arranged primers, and wherein the arrangement criteria include the following:
(i) the number or proportion of use of a degenerate base and/or universal base introduced into a primer; wherein the smaller the number or proportion of use, the higher the rank,
(ii) the number of primer patterns generated by the introduction of a degenerate base; wherein the smaller the number of patterns, the higher the rank,
(iii) the number of (A)ₙ, (T)ₙ, or (C)ₙ mononucleotide run sequences; wherein the smaller the number of the sequences, the higher the rank,
(iv) the number or proportion of consecutive nucleotides involved in the formation of a homodimer when a primer forms the homodimer; wherein the smaller the number or proportion, the higher the rank,
(v) a hairpin structure-forming free energy value (ΔG value); wherein the larger the free energy value, the higher the rank,
(vi) a Tm value; wherein the higher the Tm value, the higher the rank,
(vii) a GC content; wherein the more the GC content, the higher the rank, and
(Viii) a length; wherein the shorter the length, the higher the rank.

In the present embodiment, primers having the same start position for designing a primer, among the primers included in the primer candidate group, are arranged by giving ranks, and then the highest-ranked primers are selected. Since the primers having the same start position for designing a primer among the primers included in the primer candidate group would be present according to the primer design position within the mutant target nucleic acid sequence, the highest-ranked primers are selected according to the design position.

According to the present embodiment, in the step (f-1), the primers having the same start position for designing a primer among the primers included in the primer candidate group are arranged to satisfy at least one (specifically, arrangement criterion (i)), specifically at least two, more specifically at least three, at least four, at least five, or at least six, at least seven, or at least eight, and most specifically eight of arrangement criteria considering ranks.

There may be largely two manners in the arrangement of the primers having the same start position for designing a primer among the primers included in the primer candidate group.

According to a first manner, the at least two arrangement criteria are different in criticality, and the primers may be arranged to satisfy the arrangement criterion with the highest criticality (e.g., arrangement criterion (i)).

If multiple primers satisfy the arrangement criterion with the highest criticality, the primers may be arranged to satisfy the next-order arrangement criterion.

For example, if the criticality of the arrangement criteria is the order of arrangement criteria (i), (ii), (iii), (iv), (v), (vi), (vii) and (viii), and three primers satisfy arrangement criterion (i), the three primers are arranged according to arrangement criterion (ii). If three primers satisfy arrangement criterion (ii), the primers may be arranged to satisfy arrangement criterion (iii) to select the highest-ranked primers as a primer candidate group.

According to a second manner, different weights are assigned to the arrangement criteria and scores are assigned to values (or value ranges) in each of the arrangement criteria, and thus, the total score of each of the primers may be obtained considering ranks, and considering the calculated total scores, the primers may be arranged, and the highest-ranked primers may be selected as the primer candidate group through the ranks according to total scores.

According to an embodiment of the present invention, the at least two arrangement criteria are different in criticality, and the method of the present invention further includes a step of arranging the primers having the same start position for designing a primer among the primers included in the primer candidate group to satisfy the at least two arrangement criteria considering the criticality.

Step (g): Providing combination of probe and primer

A combination of a probe and primers is provided by combining the third probe candidate group and the primer candidate group.

In cases where the detection of the nucleotide mutation of interest within the target nucleic acid sequence is performed using an appropriate combination of a probe and primers, such as the PTOCE, VD-PTOCE, and TaqMan probe methods, the partnership between the probe and the primers in the detection of the nucleotide mutation of interest is also important although excellent properties of the probe and the primers per se are important.

For example, primers need to be capable of producing an amplicon having an appropriate size (specifically, 100-1000 nucleotides, more specifically 200-800, still more specifically 300-700, still more specifically 300-500, and still more specifically 300-400 nucleotides), while located upstream and downstream on the basis of the selected probe.

Also, a primer has preferably no interference with a probe. A representative example of such interference is dimer formation. Although a primer and a probe have excellent properties, the primer may not be appropriate when the primer forms a heterodimer with the probe.

A primer preferably has a lower Tm value than a probe. For example, the Tm value of a primer is preferably in the range of [55° C. to (Tm of the probe minus 10° C.)° C.] with respect to a probe.

According to an embodiment of the present invention, the highest-ranked probe is selected among the probes included in the third probe candidate group and a primer suitable for the highest-ranked probe is selected among the primers included in the primer candidate group. The term “suitable” means that the probe has at least one of the following characteristics: with respect to the selected probe, a primer does not form a heterodimer together with the probe, a primer produces an amplicon with a desired size, and a primer has a Tm value of [55° C. to (Tm of the probe minus 10° C.)° C.].

For example, in cases where the third probe candidate group including probes selected according to the present invention and the primer candidate group including primers are provided, the highest-ranked probe is selected among the third probe candidate group and the highest-ranked primer suitable for the probe is selected from the primer candidate group. If the primer suitable for the highest-ranked probe is absent in the primer candidate group, a primer suitable for the second highest-ranked probe is selected from the primer candidate group, and a combination of this second highest-ranked probe and the primers is used as oligonucleotides for detecting a nucleotide mutation of interest in the target nucleic acid sequence.

In the present invention, the primers may be selected according to the following suitability with respect to the probe: first, when the primers form a heterodimer with the probe, the suitability of the primers can be determined to be satisfied if the proportion of consecutive nucleotides involved in the formation of the homodimer is 65% or less (specifically, 60% or less, more specifically 55% or less, still more specifically 50% or less, and still more specifically 40% or less). Second, the suitability of the primers can be determined to be satisfied if the primers produce an amplicon with an appropriate size (specifically 100-1000, more specifically 200-800, still more specifically 300-700, still more specifically 300-500, and still more specifically 300-400 nucleotides). Third, the suitability of the primers can be determined to be satisfied if the Tm value of the primers is [55° C. to (Tm of the probe minus 10° C.)° C.].

As used herein while reciting oligonucleotides, the term “preparing” includes the providing of sequence information of an oligonucleotide and the manufacture of an oligonucleotide substance.

According to an embodiment of the present invention, the probe is a tagging probe further including a tagging portion comprising a non-hybridizable nucleotide sequence to the mutant target nucleic acid sequence. The probe may be a tagging probe further including a tagging portion.

A representative example of such a tagging probe may be PTO used in the PTOCE method and PTO-NV used in the VD-PTOCE method.

As for the tagging probe, the tagging portion is designed to satisfy the design conditions for a tagging portion in addition to the design conditions for a probe.

According to an embodiment of the present invention, the tagging portion of the tagging probe is designed to satisfy at least one of the following conditions: (i) a tagging portion length of 6-30 nucleotides; (ii) inclusion of a mismatching sequence accounting for 30% or more of the tagging portion length; and (iii) inclusion of a mismatching sequence accounting for 40% or more of the length of the 3′-end portion of the tagging portion.

The length of the tagging portion is specifically 6-20 nucleotides, 10-nucleotides, 10-20 nucleotides, 12-30 nucleotides, or 12-20 nucleotides.

The tagging portion is sufficiently non-complementary to a particular region of the mutant target nucleic acid sequence hybridizing with the tagging probe, so that the tagging portion needs to be not hybridized with the particular region under conditions in which a probe (targeting portion) of the tagging probe is hybridized. Specifically, the tagging portion includes a mismatching sequence of 30% or more, 40% or more, or 50% or more of the length thereof. Specifically, the 3′-end portion of the tagging portion includes a mismatching sequence of 40% or more or 50% or more of the length thereof.

The length of the 3′-end portion of the tagging portion may have a length of, for example, 3-8 nucleotides, 3-7 nucleotides, 3-6 nucleotides, 3-5 nucleotides, 3-4 nucleotides, 4-8 nucleotides, 4-7 nucleotides, 4-6 nucleotides, or 4-5 nucleotides.

### Storage Medium, Device, and Program

In another aspect of the present invention, there is provided a computer readable storage medium containing instructions to configure a processor to perform a method for preparing an oligonucleotide used to detect a nucleotide mutation of interest in a target nucleic acid sequence, the method comprising: (a) inputting information about a wild-type target nucleic acid sequence and a nucleotide mutation of interest; wherein the wild-type target nucleic acid sequence is a target nucleic acid sequence not comprising the nucleotide mutation of interest, the information includes information about (i) the wild-type target nucleic acid sequence, (ii) a position of the nucleotide mutation of interest occurring in the wild-type target nucleic acid sequence and (iii) wild-type and mutant bases at the position of the nucleotide mutation of interest, and wherein the position of the nucleotide mutation of interest is expressed as a start position and an end position, (b) providing the wild-type target nucleic acid sequence and a mutant target nucleic acid sequence by using the input information; wherein the mutant target nucleic acid sequence is a target nucleic acid sequence comprising the nucleotide mutation of interest, and the wild-type and mutant target nucleic acid sequences include forward and reverse wild-type and mutant target nucleic acid sequences, respectively, (c) providing a first oligonucleotide candidate group for the mutant target nucleic acid sequence by designing oligonucleotides used to detect the nucleotide mutation of interest in a predetermined region comprising the nucleotide mutation of interest within the mutant target nucleic acid sequence; (d) providing oligonucleotides satisfying the following selection criteria as a second oligonucleotide candidate group for the mutant target nucleic acid sequence by analyzing matching of the wild-type target nucleic acid sequence with the oligonucleotides included in the first oligonucleotide candidate group for the mutant target nucleic acid sequence; wherein the selection criteria include that (i) the number of mismatches between the wild-type target nucleic acid sequence and a predetermined region at the 5′-end, middle or 3′-end of an oligonucleotide is one or more; or (ii) the ratio of mismatches between the wild-type target nucleic acid sequence and an oligonucleotide is a predetermined value or more; and (e) providing a third oligonucleotide candidate group by selecting oligonucleotides from the second oligonucleotide candidate group for the mutant target nucleic acid sequence; wherein the third oligonucleotide candidate group is used to detect the nucleotide mutation of interest in the target nucleic acid sequence.

In still another aspect of the present invention, there is provided a computer program to be stored on a computer readable storage medium, to configure a processor to perform a method for preparing an oligonucleotide used to detect a nucleotide mutation of interest in a target nucleic acid sequence, the method comprising: (a) inputting information about a wild-type target nucleic acid sequence and a nucleotide mutation of interest; wherein the wild-type target nucleic acid sequence is a target nucleic acid sequence not comprising the nucleotide mutation of interest, the information includes information about (i) the wild-type target nucleic acid sequence, (ii) a position of the nucleotide mutation of interest occurring in the wild-type target nucleic acid sequence and (iii) wild-type and mutant bases at the position of the nucleotide mutation of interest, and wherein the position of the nucleotide mutation of interest is expressed as a start position and an end position, (b) providing the wild-type target nucleic acid sequence and a mutant target nucleic acid sequence by using the input information; wherein the mutant target nucleic acid sequence is a target nucleic acid sequence comprising the nucleotide mutation of interest, and the wild-type and mutant target nucleic acid sequences include forward and reverse wild-type and mutant target nucleic acid sequences, respectively, (c) providing a first oligonucleotide candidate group for the mutant target nucleic acid sequence by designing oligonucleotides used to detect the nucleotide mutation of interest in a predetermined region comprising the nucleotide mutation of interest within the mutant target nucleic acid sequence; (d) providing oligonucleotides satisfying the following selection criteria as a second oligonucleotide candidate group for the mutant target nucleic acid sequence by analyzing matching of the wild-type target nucleic acid sequence with the oligonucleotides included in the first oligonucleotide candidate group for the mutant target nucleic acid sequence; wherein the selection criteria include that (i) the number of mismatches between the wild-type target nucleic acid sequence and a predetermined region at the 5′-end, middle or 3′-end of an oligonucleotide is one or more; or (ii) the ratio of mismatches between the wild-type target nucleic acid sequence and an oligonucleotide is a predetermined value or more; and (e) providing a third oligonucleotide candidate group by selecting oligonucleotides from the second oligonucleotide candidate group for the mutant target nucleic acid sequence; wherein the third oligonucleotide candidate group is used to detect the nucleotide mutation of interest in the target nucleic acid sequence.

In another aspect of the present invention, there is provided a device for preparing an oligonucleotide used to detect a nucleotide mutation of interest in a target nucleic acid sequence, comprising (a) a computer processor and (b) a computer readable storage medium of the present invention coupled to the computer processor.

Since the storage medium, the device, and the computer program of the prevent invention are intended to perform the present methods described as above in a computer, the common descriptions among them are omitted in order to avoid undue redundancy leading to the complexity of this specification.

The program instructions are operative, when performed by the processor, to cause the processor to perform the present method described above. The program instructions for performing the method of preparing an oligonucleotide used to detect a nucleotide mutation of interest in a target nucleic acid sequence may comprise the following instructions: (i) an instruction to input information about a wild-type target nucleic acid sequence and a nucleotide mutation of interest; (ii) an instruction to provide the wild-type target nucleic acid sequence and a mutant target nucleic acid sequence by using the input information; (iii) an instruction to provide a first oligonucleotide candidate group for the mutant target nucleic acid sequence by designing oligonucleotides used to detect the nucleotide mutation of interest in a predetermined region comprising the nucleotide mutation of interest within the mutant target nucleic acid sequence; (iv) an instruction to provide oligonucleotides satisfying a predetermined selection criteria as a second oligonucleotide candidate group for the mutant target nucleic acid sequence by analyzing matching of the wild-type target nucleic acid sequence with the oligonucleotides included in the first oligonucleotide candidate group for the mutant target nucleic acid sequence; and (v) an instruction to provide (e.g., to display on an output device) a third oligonucleotide candidate group by selecting oligonucleotides from the second oligonucleotide candidate group for the mutant target nucleic acid sequence.

The method of the present invention is implemented in a processor, wherein the processor may be a processor in a stand-alone computer, a network attached computer, or a data acquisition device, such as a real-time PCR device.

The types of the computer readable storage medium include various storage media, for example, CD-R, CD-ROM, DVD, flash memory, floppy disk, hard drive, portable HDD, USB, magnetic tape, MINIDISC, nonvolatile memory card, EEPROM, optical disk, optical storage medium, RAM, ROM, system memory and web server, but are not limited thereto.

The oligonucleotides used to detect a nucleotide mutation of interest in a target nucleic acid sequence may be provided in various manners. For example, the oligonucleotides may be provided to a separate system, such as a desktop computer system, via a network connection (e.g., LAN, VPN, intranet, and internet) or a direct connection (e.g., USB or other direct wired or wireless connection), or may be provided on a portable medium such as a CD, DVD, floppy disk and portable HDD. Similarly, the oligonucleotides may be provided to a server system via a network connection (e.g., LAN, VPN, internet, intranet and wireless communication network) to a client, such as a notebook or a desktop computer system.

The instructions to configure the processor to perform the present invention may be included in a logic system. The instructions may be downloaded and stored in a memory module (e.g., hard drive or other memory, such as a local or attached RAM or ROM), although the instructions can be provided on any software storage medium (e.g., portable HDD, USB, floppy disk, CD and DVD). A computer code for implementing the present invention may be implemented in a variety of coding languages, such as C, C++, Java, Visual Basic, VBScript, JavaScript, Perl and XML. In addition, a variety of languages and protocols may be used in external and internal storage and transmission of data and commands according to the present invention.

The computer processor may be prepared in such a manner that a single processor can do several performances. Alternatively, the processor unit may be prepared in such a manner that several processors do the several performances, respectively.

The features and advantages of the present invention are summarized as follows:
(a) The conventional methods had a problem in that various design rules and modules need to be developed for respective considerations including the type of nucleotide mutation, the size of the mutation, and whether a target to be detected is a wild-type and/or mutant sequence, in order to design oligonucleotides for detecting a nucleotide mutation of interest, such as a substitution, an inversion, an insertion, a deletion, a duplication, or a complex thereof. Moreover, such conventional methods had a problem in that the designing of oligonucleotides without considering of sequence contents fails to distinguish between a wild-type target nucleic acid sequence and a mutant target nucleic acid sequence, and had a disadvantage in that the design rules become complicated when considering the contents of such sequences.
(b) On the contrary, the present invention can effectively prepare oligonucleotides used to detect a nucleotide mutation of interest within a target nucleic acid sequence, by inputting information about a wild-type target nucleic acid sequence and the nucleotide mutation of interest, providing wild-type and mutant target nucleic acid sequences through the use of the input information, designing oligonucleotides for the mutant target nucleic acid sequence, and analyzing the matching between the designed oligonucleotides and the wild-type target nucleic acid sequence to select and provide oligonucleotides satisfying predetermined selection criteria.
(c) Furthermore, the present invention can prepare oligonucleotides capable of detecting a nucleotide mutation on the basis of integrated design rules, without the need to develop detailed design rules and modules considering the type of nucleotide mutation, the size of the mutation, whether a target to be detected is a wild-type and/or mutant sequence, sequence contents, and the like.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Example 1: Preparation of Oligonucleotides Used to Detect Mutation (T112C, Substitution of T with C) in Human Gene ApoE

Oligonucleotides used to detect a substitution mutation (T112C) in the human gene ApoE were prepared by performing a program (HMOD) for preparing oligonucleotides used to detect a nucleotide mutation of interest in a target nucleic acid sequence.

Inputting of Information about Wild-Type Target Nucleic Acid Sequence of ApoE Gene and Substitution Mutation (T112C)

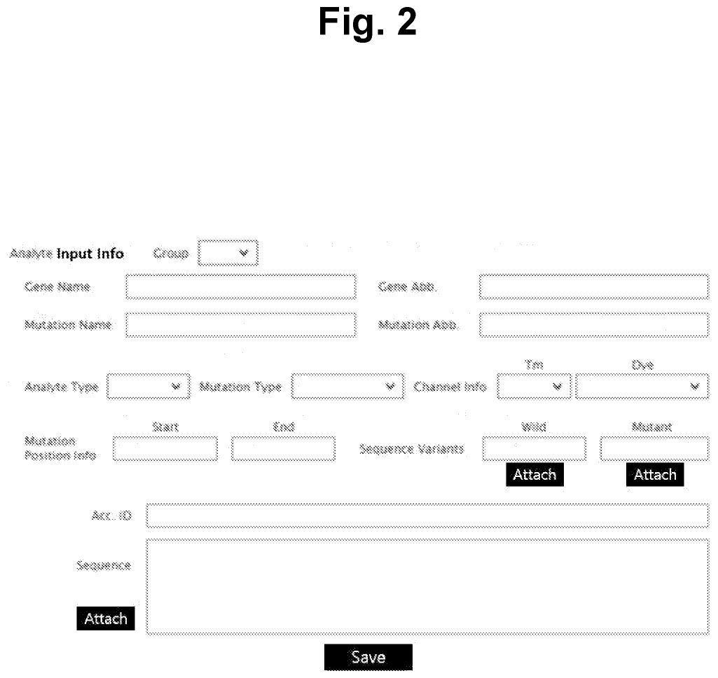

Information about a wild-type target nucleic acid sequence of ApoE gene and a substitution mutation (T112C) for running HMOD program was input through a user interface (UI) window (FIG. 2) of HMOD program. Specifically, in the UI of FIG. 2, ApoE was entered in Gene Name, ApoE in Gene Abb., T112C in Mutation Name, T112C in Mutation Abb., Mutant in Analyte Type, substitution in Mutation Type, 501 in Start and 501 in End of Mutation Position Info, T in Wild and C in Mutant of Sequence Variants, NC_018930.2 in Acc.ID, and the sequence shown in Table 1 below in Sequence, and then the HMOD program was run. The start position and end position of the nucleotide mutation of interest entered in Start and End of Mutation Position Info indicate positions in the wild-type target nucleic acid sequence shown in Table 1 below.

TABLE 1
Sequence (SEQ ID NO: 4)
(5′ to 3′)

CTCTGTCCTTCCCTAGCTCTTTTATATAGAGACAGAGAGATGGGGTCTCACTGTGTTGCCCAGGCT
GGTCTTGAACTTCTGGGCTCAAGCGATCCTCCCGCCTCGGCCTCCCAAAGTGCTGGGATTAGAGG
CATGAGCCACCTTGCCCGGCCTCCTAGCTCCTTCTTCGTCTCTGCCTCTGCCCTCTGCATCTGCTC
TCTGCATCTGTCTCTGTCTCCTTCTCTCGGCCTCTGCCCCGTTCCTTCTCTCCCTCTTGGGTCTCTC
TGGCTCATCCCCATCTCGCCCGCCCCATCCCAGCCCTTCTCCCCGCCTCCCACTGTGCGACACCCT
CCCGCCCTCTCGGCCGCAGGGCGCTGATGGACGAGACCATGAAGGAGTTGAAGGCCTACAAATCG
GAACTGGAGGAACAACTGACCCCGGTGGCGGAGGAGACGCGGGCACGGCTGTCCAAGGAGCTG
CAGGCGGCGCAGGCCCGGCTGGGCGCGGACATGGAGGACGTGTGCGGCCGCCTGGTGCAGTAC
CGCGGCGAGGTGCAGGCCATGCTCGGCCAGAGCACCGAGGAGCTGCGGGTGCGCCTCGCCTCCC
ACCTGCGCAAGCTGCGTAAGCGGCTCCTCCGCGATGCCGATGACCTGCAGAAGCGCCTGGCAGT
GTACCAGGCCGGGGCCCGCGAGGGCGCCGAGCGCGGCCTCAGCGCCATCCGCGAGCGCCTGGG
GCCCCTGGTGGAACAGGGCCGCGTGCGGGCCGCCACTGTGGGCTCCCTGGCCGGCCAGCCGCTA
CAGGAGCGGGCCCAGGCCTGGGGCGAGCGGCTGCGCGCGCGGATGGAGGAGATGGGCAGCCGG
ACCCGCGACCGCCTGGACGAGGTGAAGGAGCAGGTGGCGGAGGTGCGCGCCAAGCTGGAGGAG
CAGGCCCAGCAGATACGCCTGCAGGCCGAGGCCTTCCAGGCCCGCCTCAAGAGCTGGTTCGAGC
CCCTGGTGGAAGACATGCAGCGCCAGTGGGCCGGGCTGGTGGAGAAGGTGCAGGCTGCCGTGG
GCACCAGCGCCGCCCCTGTGCCCAGCGACAATCACTGAACGCCGAAGCCTGCAGCCATGCGACCC
CACGCCACCCCGTGCCTCCTGCCTCCGCGCAGCCTGCAGCGGGAG

The T base written in bold in Table 1 corresponds to the 501st position in the sequence, and the substitution mutation T112C represents a mutation in which the T base is substituted with the C base at the 501st position in the sequence.

The HMOD program was run to proceed in the following order:

### Providing Wild-Type and Mutant Target Nucleic Acid Sequences

Oligonucleotides were designed using the input information, and forward and reverse wild-type and mutant target nucleic acid sequences were provided to be used as templates for confirming that the oligonucleotides designed for the mutant target nucleic acid sequence could be distinguishably detected from the wild-type nucleic acid sequences.

The forward and reverse wild-type and mutant target nucleic acid sequences were summarized in Table 2 below.

TABLE 2

| Type | Sequence |
|---|---|
| Forward wild-type | CTCTGTCCTTCCCTAGCTCTTTTATATAGAGACAGAGAGATGGGGTCTCA |
| target nucleic acid | CTGTGTTGCCCAGGCTGGTCTTGAACTTCTGGGCTCAAGCGATCCTCCC |
| sequence | GCCTCGGCCTCCCAAAGTGCTGGGATTAGAGGCATGAGCCACCTTGCCC |
| (SEQ ID NO: 4) | GGCCTCCTAGCTCCTTCTTCGTCTCTGCCTCTGCCCTCTGCATCTGCTCT |
| (5′ to 3′) | CTGCATCTGTCTCTGTCTCCTTCTCTCGGCCTCTGCCCCGTTCCTTCTCT |
| | CCCTCTTGGGTCTCTCTGGCTCATCCCCATCTCGCCCGCCCCATCCCAGC |
| | CCTTCTCCCCGCCTCCCACTGTGCGACACCCTCCCGCCCTCTCGGCCGCA |
| | GGGCGCTGATGGACGAGACCATGAAGGAGTTGAAGGCCTACAAATCGGA |
| | ACTGGAGGAACAACTGACCCCGGTGGCGGAGGAGACGCGGGCACGGCT |
| | GTCCAAGGAGCTGCAGGCGGCGCAGGCCCGGCTGGGCGCGGACATGGA |
| | GGACGTGTGCGGCCGCCTGGTGCAGTACCGCGGCGAGGTGCAGGCCAT |
| | GCTCGGCCAGAGCACCGAGGAGCTGCGGGTGCGCCTCGCCTCCCACCT |
| | GCGCAAGCTGCGTAAGCGGCTCCTCCGCGATGCCGATGACCTGCAGAAG |
| | CGCCTGGCAGTGTACCAGGCCGGGGCCCGCGAGGGCGCCGAGCGCGGC |
| | CTCAGCGCCATCCGCGAGCGCCTGGGGCCCCTGGTGGAACAGGGCCGC |
| | GTGCGGGCCGCCACTGTGGGCTCCCTGGCCGGCCAGCCGCTACAGGAG |
| | CGGGCCCAGGCCTGGGGCGAGCGGCTGCGCGCGCGGATGGAGGAGAT |
| | GGGCAGCCGGACCCGCGACCGCCTGGACGAGGTGAAGGAGCAGGTGGC |
| | GGAGGTGCGCGCCAAGCTGGAGGAGCAGGCCCAGCAGATACGCCTGCA |
| | GGCCGAGGCCTTCCAGGCCCGCCTCAAGAGCTGGTTCGAGCCCCTGGTG |
| | GAAGACATGCAGCGCCAGTGGGCCGGGCTGGTGGAGAAGGTGCAGGCT |
| | GCCGTGGGCACCAGCGCCGCCCCTGTGCCCAGCGACAATCACTGAACGC |
| | CGAAGCCTGCAGCCATGCGACCCCACGCCACCCCGTGCCTCCTGCCTCC |
| | GCGCAGCCTGCAGCGGGAG |
| Reverse wild-type | CTCCCGCTGCAGGCTGCGCGGAGGCAGGAGGCACGGGGTGGCGTGGG |
| target nucleic acid | GTCGCATGGCTGCAGGCTTCGGCGTTCAGTGATTGTCGCTGGGCACAGG |
| sequence | GGCGGCGCTGGTGCCCACGGCAGCCTGCACCTTCTCCACCAGCCCGGCC |
| (SEQ ID NO: 5) | CACTGGCGCTGCATGTCTTCCACCAGGGGCTCGAACCAGCTCTTGAGGC |
| (5′ to 3′) | GGGCCTGGAAGGCCTCGGCCTGCAGGCGTATCTGCTGGGCCTGCTCCTC |
| | CAGCTTGGCGCGCACCTCCGCCACCTGCTCCTTCACCTCGTCCAGGCGG |
| | TCGCGGGTCCGGCTGCCCATCTCCTCCATCCGCGCGCGCAGCCGCTCGC |
| | CCCAGGCCTGGGCCCGCTCCTGTAGCGGCTGGCCGGCCAGGGAGCCCA |
| | CAGTGGCGGCCCGCACGCGGCCCTGTTCCACCAGGGGCCCCAGGCGCT |
| | CGCGGATGGCGCTGAGGCCGCGCTCGGCGCCCTCGCGGGCCCCGGCCT |
| | GGTACACTGCCAGGCGCTTCTGCAGGTCATCGGCATCGCGGAGGAGCCG |
| | CTTACGCAGCTTGCGCAGGTGGGAGGCGAGGCGCACCCGCAGCTCCTC |
| | GGTGCTCTGGCCGAGCATGGCCTGCACCTCGCCGCGGTACTGCACCAGG |
| | CGGCCGCACACGTCCTCCATGTCCGCGCCCAGCCGGGCCTGCGCCGCCT |
| | GCAGCTCCTTGGACAGCCGTGCCCGCGTCTCCTCCGCCACCGGGGTCAG |
| | TTGTTCCTCCAGTTCCGATTTGTAGGCCTTCAACTCCTTCATGGTCTCGTC |
| | CATCAGCGCCCTGCGGCCGAGAGGGCGGGAGGGTGTCGCACAGTGGGA |
| | GGCGGGGAGAAGGGCTGGGATGGGGGGGGCGAGATGGGGATGAGCCA |
| | GAGAGACCCAAGAGGGAGAGAAGGAACGGGGCAGAGGCCGAGAGAAG |
| | GAGACAGAGACAGATGCAGAGAGCAGATGCAGAGGGCAGAGGCAGAGA |
| | CGAAGAAGGAGCTAGGAGGCCGGGCAAGGTGGCTCATGCCTCTAATCCC |
| | AGCACTTTGGGAGGCCGAGGCGGGAGGATCGCTTGAGCCCAGAAGTTCA |
| | AGACCAGCCTGGGCAACACAGTGAGACCCCATCTCTCTGTCTCTATATAA |
| | AAGAGCTAGGGAAGGACAGAG |
| Forward mutant | CTCTGTCCTTCCCTAGCTCTTTTATATAGAGACAGAGAGATGGGGTCTCA |
| target nucleic acid | CTGTGTTGCCCAGGCTGGTCTTGAACTTCTGGGCTCAAGCGATCCTCCC |
| sequence | GCCTCGGCCTCCCAAAGTGCTGGGATTAGAGGCATGAGCCACCTTGCCC |
| (SEQ ID NO: 6) | GGCCTCCTAGCTCCTTCTTCGTCTCTGCCTCTGCCCTCTGCATCTGCTCT |
| (5′ to 3′) | CTGCATCTGTCTCTGTCTCCTTCTCTCGGCCTCTGCCCCGTTCCTTCTCT |
| | CCCTCTTGGGTCTCTCTGGCTCATCCCCATCTCGCCCGCCCCATCCCAGC |
| | CCTTCTCCCCGCCTCCCACTGTGCGACACCCTCCCGCCCTCTCGGCCGCA |
| | GGGCGCTGATGGACGAGACCATGAAGGAGTTGAAGGCCTACAAATCGGA |
| | ACTGGAGGAACAACTGACCCCGGTGGCGGAGGAGACGCGGGCACGGCT |
| | GTCCAAGGAGCTGCAGGCGGCGCAGGCCCGGCTGGGCGCGGACATGGA |
| | GGACGTGCGCGGCCGCCTGGTGCAGTACCGCGGCGAGGTGCAGGCCAT |
| | GCTCGGCCAGAGCACCGAGGAGCTGCGGGTGCGCCTCGCCTCCCACCT |
| | GCGCAAGCTGCGTAAGCGGCTCCTCCGCGATGCCGATGACCTGCAGAAG |
| | CGCCTGGCAGTGTACCAGGCCGGGGCCCGCGAGGGCGCCGAGCGCGGC |
| | CTCAGCGCCATCCGCGAGCGCCTGGGGCCCCTGGTGGAACAGGGCCGC |
| | GTGCGGGCCGCCACTGTGGGCTCCCTGGCCGGCCAGCCGCTACAGGAG |
| | CGGGCCCAGGCCTGGGGCGAGCGGCTGCGCGCGCGGATGGAGGAGAT |
| | GGGCAGCCGGACCCGCGACCGCCTGGACGAGGTGAAGGAGCAGGTGGC |
| | GGAGGTGCGCGCCAAGCTGGAGGAGCAGGCCCAGCAGATACGCCTGCA |
| | GGCCGAGGCCTTCCAGGCCCGCCTCAAGAGCTGGTTCGAGCCCCTGGTG |
| | GAAGACATGCAGCGCCAGTGGGCCGGGCTGGTGGAGAAGGTGCAGGCT |
| | GCCGTGGGCACCAGCGCCGCCCCTGTGCCCAGCGACAATCACTGAACGC |
| | CGAAGCCTGCAGCCATGCGACCCCACGCCACCCCGTGCCTCCTGCCTCC |
| | GCGCAGCCTGCAGCGGGAG |
| Reverse mutant | CTCCCGCTGCAGGCTGCGCGGAGGCAGGAGGCACGGGGGGCGTGGG |
| target nucleic acid | GTCGCATGGCTGCAGGCTTCGGCGTTCAGTGATTGTCGCTGGGCACAGG |
| sequence | GGCGGCGCTGGTGCCCACGGCAGCCTGCACCTTCTCCACCAGCCCGGCC |
| (SEQ ID NO: 7) | CACTGGCGCTGCATGTCTTCCACCAGGGGCTCGAACCAGCTCTTGAGGC |
| (5′ to 3′) | GGGCCTGGAAGGCCTCGGCCTGCAGGCGTATCTGCTGGGCCTGCTCCTC |
| | CAGCTTGGCGCGCACCTCCGCCACCTGCTCCTTCACCTCGTCCAGGCGG |
| | TCGCGGGTCCGGCTGCCCATCTCCTCCATCCGCGCGCGCAGCCGCTCGC |
| | CCCAGGCCTGGGCCCGCTCCTGTAGCGGCTGGCCGGCCAGGGAGCCCA |
| | CAGTGGCGGCCCGCACGCGGCCCTGTTCCACCAGGGGCCCCAGGCGCT |
| | CGCGGATGGCGCTGAGGCCGCGCTCGGCGCCCTCGCGGGCCCCGGCCT |
| | GGTACACTGCCAGGCGCTTCTGCAGGTCATCGGCATCGCGGAGGAGCCG |
| | CTTACGCAGCTTGCGCAGGTGGGAGGCGAGGCGCACCCGCAGCTCCTC |
| | GGTGCTCTGGCCGAGCATGGCCTGCACCTCGCCGCGGTACTGCACCAGG |
| | CGGCCGCGCACGTCCTCCATGTCCGCGCCCAGCCGGGCCTGCGCCGCCT |
| | GCAGCTCCTTGGACAGCCGTGCCCGCGTCTCCTCCGCCACCGGGGTCAG |
| | TTGTTCCTCCAGTTCCGATTTGTAGGCCTTCAACTCCTTCATGGTCTCGTC |
| | CATCAGCGCCCTGCGGCCGAGAGGGGGGAGGGTGTCGCACAGTGGGA |
| | GGCGGGGAGAAGGGCTGGGATGGGGGGGGCGAGATGGGGATGAGCCA |
| | GAGAGACCCAAGAGGGAGAGAAGGAACGGGGCAGAGGCCGAGAGAAG |
| | GAGACAGAGACAGATGCAGAGAGCAGATGCAGAGGGCAGAGGCAGAGA |
| | CGAAGAAGGAGCTAGGAGGCCGGGCAAGGTGGCTCATGCCTCTAATCCC |
| | AGCACTTTGGGAGGCCGAGGCGGGAGGATCGCTTGAGCCCAGAAGTTCA |
| | AGACCAGCCTGGGCAACACAGTGAGACCCCATCTCTCTGTCTCTATATAA |
| | AAGAGCTAGGGAAGGACAGAG |

### Providing a First Probe Candidate Group

Probes used to detect the nucleotide mutation of interest within the mutant target nucleic acid sequence were designed to satisfy the following conditions: (i) a length of 10-60 nucleotides; (ii) a Tm value of 50-85° C.; (iii) exclusion of G-run sequence with at least three Gs; and (iv) a GC content of 40% or more of the 5′-end portion.

Specifically, probes were designed according to lengths (by length) (predetermined lengths: 17 to 35 mer) at each position from position 499, which is present 2 bp upstream from position 501 as the start position of the nucleotide mutation of interest (the C base), to position 501 as the end position of the nucleotide mutation of interest within the provided forward mutant target nucleic acid sequence, and these probes were provided as the first probe candidate group. In addition, probes were designed according to lengths (by length) (predetermined lengths: 17 to 35 mer) at each position from position 637, which is present 2 bp upstream from position 639 as the start position of the nucleotide mutation of interest (the G base), to position 639 as the end position of the nucleotide mutation of interest within the provided reverse mutant target nucleic acid sequence, and these probes were provided as the first probe candidate group. The number of probes designed for the forward and reverse mutant target nucleic acid sequences was 114, and of these, forward probes having 19 and 20 mer were summarized in Table 3 below.

TABLE 3

| Strand | Sequence | Start | End | Note |
|---|---|---|---|---|
| Forward | TGCGCGGCCGCCTGGTGCA | 499 | 517 | SEQ ID NO: 8 |
| | TGCGCGGCCGCCTGGTGCAG | 499 | 518 | SEQ ID NO: 9 |
| | GCGCGGCCGCCTGGTGCAG | 500 | 518 | SEQ ID NO: 10 |
| | GCGCGGCCGCCTGGTGCAGT | 500 | 519 | SEQ ID NO: 11 |
| | CGCGGCCGCCTGGTGCAGT | 501 | 519 | SEQ ID NO: 12 |
| | CGCGGCCGCCTGGTGCAGTA | 501 | 520 | SEQ ID NO: 13 |

### Providing a Second Probe Candidate Group

Probes satisfying the following selection criteria were provided as a second probe candidate group for the mutant target nucleic acid sequence by analyzing matching of the wild-type target nucleic acid sequence with the probes included in the first probe candidate group for the mutant target nucleic acid sequence. The selection criteria were as follows: (i) the number of mismatches between the wild-type target nucleic acid sequence and the 5′-end of a probe is one; or (ii) the ratio of the number of minimum mismatches between the wild-type target nucleic acid sequence and a probe is 30% or more compared to the probe sequence.

The number of probes satisfying the selection criteria among the probes included in the first probe candidate group was 10 and, of these, only five probes were summarized in Table 4 below.

TABLE 4

| Strand | Sequence | Start | End | Note |
|---|---|---|---|---|
| Forward | CGCGGCCGCCTGGTGCA | 501 | 517 | SEQ ID NO: 14 |
| | CGCGGCCGCCTGGTGCAG | 501 | 518 | SEQ ID NO: 15 |
| | CGCGGCCGCCTGGTGCAGT | 501 | 519 | SEQ ID NO: 16 |
| | CGCGGCCGCCTGGTGCAGTA | 501 | 520 | SEQ ID NO: 17 |
| | CGCGGCCGCCTGGTGCAGTAC | 501 | 521 | SEQ ID NO: 18 |

Then, the 10 probes were arranged by giving ranks according to the arrangement criteria below: (i) the position of the minimum mismatch in a probe as a result of the matching analysis; wherein the closer the position of the minimum mismatch is to the 5′-end of the probe, the higher the rank, (ii) the number of minimum mismatches in a probe as a result of the matching analysis; wherein the larger the number of minimum mismatches, the higher the rank, (iii) a Tm value of a probe; wherein the higher the Tm value, the higher the rank, (iv) a GC content in a predetermined region of the 5′-end of a probe; wherein the higher the GC content, the higher the rank, (v) the number of consecutive G bases included in a probe; wherein the smaller the number of consecutive G bases, the higher the rank, (vi) the number or proportion of consecutive nucleotides involved in the formation of a homodimer when a probe forms the homodimer; wherein the smaller the number or proportion, the higher the rank, (vii) a hairpin structure-forming free energy value (ΔG value); wherein the larger the free energy value, the higher the rank, and (viii) a length; wherein the shorter the length, the higher the rank.

### Providing a Third Probe Candidate Group

Out of probes having the same start position for designing a probe among the probes included in the second probe candidate group, the highest-ranked probes with respect to ranking according to the arrangement criteria were provided as the third probe candidate group. Among the second probe candidate group comprising the 10 probes, two probes were selected as the third probe candidate group, and the results were summarized in Table 5 below.

TABLE 5

| Strand | Sequence | Note |
|---|---|---|
| Reverse | GCACGTCCTCCATGTCCGC | SEQ ID NO: 19 |
| Forward | CGCGGCCGCCTGGTGCAGT | SEQ ID NO: 16 |

### Providing a Primer Candidate Group

Primer candidate group for the mutant target nucleic acid sequences were provided by designing primers to amplify a predetermined region comprising the nucleotide mutation of interest within the mutant target nucleic acid sequence. Specifically, a primer design range to be designed to amplify a predetermined region comprising the nucleotide mutation of interest was selected in a region from 300 bp upstream of the start position of the nucleotide mutation of interest to 300 bp downstream of the end position of the nucleotide mutation of interest. In addition, the primers to be designed were designed to satisfy the following conditions: (i) a Tm value of 40-70° C.; (ii) a length of 15-50 bp nucleotides; and (iii) exclusion of a G-run sequence with at least five Gs.

In addition, primers having the same start position for designing a primer among the primers included in the primer candidate group were arranged by giving ranks according to the arrangement criteria, and each highest-ranked oligonucleotide was selected: (i) the number or proportion of use of a degenerate base and/or universal base introduced into a primer; wherein the smaller the number or proportion of use, the higher the rank, (ii) the number of primer patterns generated by the introduction of a degenerate base; wherein the smaller the number of patterns, the higher the rank, (iii) the number of (A)ₙ, (T)ₙ, or (C)ₙ mononucleotide run sequences; wherein the smaller the number of the sequences, the higher the rank, (iv) the number or proportion of consecutive nucleotides involved in the formation of a homodimer when the primer forms the homodimer; wherein the smaller the number or proportion, the higher the rank, (v) a hairpin structure-forming free energy value (ΔG value); wherein the larger the free energy value, the higher the rank, (vi) a Tm value; wherein the higher the Tm value, the higher the rank, (vii) a GC content; wherein the more the GC content, the higher the rank, and (viii) a length; wherein the shorter the length, the higher the rank.

As a result, 12289 primers were designed as the primer candidate group, and from primers having the same start position for designing a primer among the designed primers, the 103 highest-ranked primers were selected, and examples of the selected highest-ranked primers were summarized in Table 6 below.

TABLE 6

| Strand | Sequence | Position | Note |
|---|---|---|---|
| Forward | TCTCTCGGCCTCTGCCIIIIICCTTCTCTC | 220-249 | SEQ ID NO: 20 |
| Forward | ATCCCAGCCCTTCTCCCIIIIICCCACTGTG | 291-321 | SEQ ID NO: 21 |
| Forward | TGGACGAGACCATGAAGIIIIIGAAGGCCTA | 358-388 | SEQ ID NO: 22 |
| Reverse | GTGGGAGGCGAGGCGIIIIIGCAGCTCCT | 553-581 | SEQ ID NO: 23 |
| Reverse | GCAGCTCCTCGGTGCTIIIIICGAGCATGG | 573-602 | SEQ ID NO: 24 |
| Reverse | TGGCCTGCACCTCGCIIIIITACTGCACC | 600-628 | SEQ ID NO: 25 |

### Combinations of Probe and Primers

Combinations of a probe and primers were provided by combining the selected third probe candidate group and primer candidate group. Specifically, combinations that produce 300-350 bp amplicons and have no dimer formed between a probe and a primer to be combined were created by combining the two probes and the 103 primers that had been selected, and out of these, three exemplary combinations were summarized in Table 7 below.

TABLE 7

| Combination | Type | Sequence | Note |
|---|---|---|---|
| 1 | Forward | GCAGGGCGCTGATGGIIIIIACCATGAAG | SEQ ID NO: 26 |
| | primer | | |
| | Reverse | CAGCCCTTCTCCCCGCIIIIIACTGTGCGA | SEQ ID NO: 27 |
| | primer | | |
| | Probe | GCACGTCCTCCATGTCCGC | SEQ ID NO: 19 |
| 2 | Forward | CATCCCAGCCCTTCTCCIIIIITCCCACTGT | SEQ ID NO: 28 |
| | primer | | |
| | Reverse | TGGGAGGCGAGGCGCIIIIICAGCTCCTC | SEQ ID NO: 29 |
| | primer | | |
| | Probe | CGCGGCCGCCTGGTGCAGT | SEQ ID NO: 16 |
| 3 | Forward | CCGCAGGGCGCTGATIIIIIAGACCATGA | SEQ ID NO: 30 |
| | primer | | |
| | Reverse | GCAGCTCCTCGGTGCTIIIIICGAGCATGG | SEQ ID NO: 24 |
| | primer | | |
| | Probe | GCACGTCCTCCATGTCCGC | SEQ ID NO: 19 |

### Example 2: Preparing Oligonucleotides Used to Detect Mutation (FS89, Insertion of C Base) in Human Gene HBB

Oligonucleotides used to detect an insertion mutation in human gene HBB were prepared by running the same program (HMOD) as in Example 1.

Inputting of Information about Wild-Type Target Nucleic Acid Sequence of HBB Gene and Insertion Mutation

Information about a wild-type target nucleic acid sequence of HBB gene and an insertion mutation for running HMOD program was input through a user interface (UI) window (FIG. 2) of HMOD program. Specifically, in the UI of FIG. 2, HBB was entered in Gene Name, HBB in Gene Abb., FS89 in Mutation Name, FS89 in Mutation Abb., Mutant in Analyte Type, insertion in Mutation Type, 396 in Start and 396 in End of Mutation Position Info, C in Wild and CC in Mutant of Sequence Variants, NC_046672.1 in Acc.ID, and the sequence shown in Table 8 below in Sequence, and then the HMOD program was run. The start position and end position of the nucleotide mutation of interest entered in Start and End of Mutation Position Info indicate positions in the wild-type target nucleic acid sequence shown in Table 8 below.

TABLE 8
Sequence (SEQ ID NO: 31)
(5′ to 3′)

GCAGCTTGTCACAGTGCAGCTCACTCAGTGTGGCAAAGGTGCCCTTGAGGTTGTCCAGGTGAGC
CAGGCCATCACTAAAGGCACCGAGCACTTTCTTGCCATGAGCCTTCACCTTAGGGTTGCCCATAA
CAGCATCAGGAGTGGACAGATCCCCAAAGGACTCAAAGAACCTCTGGGTCCAAGGGTAGACCAC
CAGCAGCCTAAGGGTGGGAAAATAGACCAATAGGCAGAGAGAGTCAGTGCCTATCAGAAACCCA
AGAGTCTTCTCTGTCTCCACATGCCCAGTTTCTATTGGTCTCCTTAAACCTGTCTTGTAACCTTGA
TACCAACCTGCCCAGGGCCTCACCACCAACTTCATCCACGTTCACCTTGCCCCACAGGGCAGTAA
CGGCAGACTTCTCCTCAGGAGTCAGATGCACCATGGTGTCTGTTTGAGGTTGCTAGTGAACACA
GTTGTGTCAGAAGCAAATGTAAGCAATAGATGGCTCTGCCCTGACTTTTATGCCCAGCCCTGGCT
CCTGCCCTCCCTGCTCCTGGGAGTAGATTGGCCAACCCTAGGGTGTGGCTCCACAGGGTGAGGT
CTAAGTGATGACAGCCGTACCTGTCCTTGGCTCTTCTGGCACTGGCTTAGGAGTTGGACTTCAAA
CCCTCAGCCCTCCCTCTAAGATATATCTCTTGGCCCCATACCATCAGTACAAATTGCTACT

The C base written in bold in Table 8 corresponds to the 396th position in the sequence, and the insertion mutation FS89 represents a mutation in which the C base is inserted between the 396th position and the 397th position in the sequence.

The HMOD program was run to proceed in the following order:

### Providing Wild-Type and Mutant Target Nucleic Acid Sequences

The forward and reverse wild-type and mutant target nucleic acid sequences were provided in the same manner as in the Example 1, and the results were summarized in Table 9 below.

TABLE 9

| Type | Sequence |
|---|---|
| Forward wild-type | GCAGCTTGTCACAGTGCAGCTCACTCAGTGTGGCAAAGGTGCCCTTGAG |
| target nucleic acid | GTTGTCCAGGTGAGCCAGGCCATCACTAAAGGCACCGAGCACTTTCTTG |
| sequence | CCATGAGCCTTCACCTTAGGGTTGCCCATAACAGCATCAGGAGTGGACAG |
| (SEQ ID NO: 31) | ATCCCCAAAGGACTCAAAGAACCTCTGGGTCCAAGGGTAGACCACCAGC |
| (5′ to 3′) | AGCCTAAGGGTGGGAAAATAGACCAATAGGCAGAGAGAGTCAGTGCCTA |
| | TCAGAAACCCAAGAGTCTTCTCTGTCTCCACATGCCCAGTTTCTATTGGT |
| | CTCCTTAAACCTGTCTTGTAACCTTGATACCAACCTGCCCAGGGCCTCAC |
| | CACCAACTTCATCCACGTTCACCTTGCCCCACAGGGCAGTAACGGCAGAC |
| | TTCTCCTCAGGAGTCAGATGCACCATGGTGTCTGTTTGAGGTTGCTAGTG |
| | AACACAGTTGTGTCAGAAGCAAATGTAAGCAATAGATGGCTCTGCCCTGA |
| | CTTTTATGCCCAGCCCTGGCTCCTGCCCTCCCTGCTCCTGGGAGTAGATT |
| | GGCCAACCCTAGGGTGTGGCTCCACAGGGTGAGGTCTAAGTGATGACAG |
| | CCGTACCTGTCCTTGGCTCTTCTGGCACTGGCTTAGGAGTTGGACTTCAA |
| | ACCCTCAGCCCTCCCTCTAAGATATATCTCTTGGCCCCATACCATCAGTAC |
| | AAATTGCTACT |
| Reverse wild-type | AGTAGCAATTTGTACTGATGGTATGGGGCCAAGAGATATATCTTAGAGGG |
| target nucleic acid | AGGGCTGAGGGTTTGAAGTCCAACTCCTAAGCCAGTGCCAGAAGAGCCA |
| sequence | AGGACAGGTACGGCTGTCATCACTTAGACCTCACCCTGTGGAGCCACAC |
| (SEQ ID NO: 32) | CCTAGGGTTGGCCAATCTACTCCCAGGAGCAGGGAGGGCAGGAGCCAG |
| (5′ to 3′) | GGCTGGGCATAAAAGTCAGGGCAGAGCCATCTATTGCTTACATTTGCTTC |
| | TGACACAACTGTGTTCACTAGCAACCTCAAACAGACACCATGGTGCATCT |
| | GACTCCTGAGGAGAAGTCTGCCGTTACTGCCCTGTGGGGCAAGGTGAAC |
| | GTGGATGAAGTTGGTGGTGAGGCCCTGGGCAGGTTGGTATCAAGGTTAC |
| | AAGACAGGTTTAAGGAGACCAATAGAAACTGGGCATGTGGAGACAGAGA |
| | AGACTCTTGGGTTTCTGATAGGCACTGACTCTCTCTGCCTATTGGTCTAT |
| | TTTCCCACCCTTAGGCTGCTGGTGGTCTACCCTTGGACCCAGAGGTTCTT |
| | TGAGTCCTTTGGGGATCTGTCCACTCCTGATGCTGTTATGGGCAACCCTA |
| | AGGTGAAGGCTCATGGCAAGAAAGTGCTCGGTGCCTTTAGTGATGGCCT |
| | GGCTCACCTGGACAACCTCAAGGGCACCTTTGCCACACTGAGTGAGCTG |
| | CACTGTGACAAGCTGC |
| Forward mutant | GCAGCTTGTCACAGTGCAGCTCACTCAGTGTGGCAAAGGTGCCCTTGAG |
| target nucleic acid | GTTGTCCAGGTGAGCCAGGCCATCACTAAAGGCACCGAGCACTTTCTTG |
| sequence | CCATGAGCCTTCACCTTAGGGTTGCCCATAACAGCATCAGGAGTGGACAG |
| (SEQ ID NO: 33) | ATCCCCAAAGGACTCAAAGAACCTCTGGGTCCAAGGGTAGACCACCAGC |
| (5′ to 3′) | AGCCTAAGGGTGGGAAAATAGACCAATAGGCAGAGAGAGTCAGTGCCTA |
| | TCAGAAACCCAAGAGTCTTCTCTGTCTCCACATGCCCAGTTTCTATTGGT |
| | CTCCTTAAACCTGTCTTGTAACCTTGATACCAACCTGCCCAGGGCCTCAC |
| | CACCAACTTCATCCACGTTCACCTTGCCCCACAGGGCAGTAACGGCAGAC |
| | CTTCTCCTCAGGAGTCAGATGCACCATGGTGTCTGTTTGAGGTTGCTAGT |
| | GAACACAGTTGTGTCAGAAGCAAATGTAAGCAATAGATGGCTCTGCCCTG |
| | ACTTTTATGCCCAGCCCTGGCTCCTGCCCTCCCTGCTCCTGGGAGTAGAT |
| | TGGCCAACCCTAGGGTGTGGCTCCACAGGGTGAGGTCTAAGTGATGACA |
| | GCCGTACCTGTCCTTGGCTCTTCTGGCACTGGCTTAGGAGTTGGACTTCA |
| | AACCCTCAGCCCTCCCTCTAAGATATATCTCTTGGCCCCATACCATCAGTA |
| | CAAATTGCTACT |
| Reverse mutant | AGTAGCAATTTGTACTGATGGTATGGGGCCAAGAGATATATCTTAGAGGG |
| target nucleic acid | AGGGCTGAGGGTTTGAAGTCCAACTCCTAAGCCAGTGCCAGAAGAGCCA |
| sequence | AGGACAGGTACGGCTGTCATCACTTAGACCTCACCCTGTGGAGCCACAC |
| (SEQ ID NO: 34) | CCTAGGGTTGGCCAATCTACTCCCAGGAGCAGGGAGGGCAGGAGCCAG |
| (5′ to 3′) | GGCTGGGCATAAAAGTCAGGGCAGAGCCATCTATTGCTTACATTTGCTTC |
| | TGACACAACTGTGTTCACTAGCAACCTCAAACAGACACCATGGTGCATCT |
| | GACTCCTGAGGAGAAGGTCTGCCGTTACTGCCCTGTGGGGCAAGGTGAA |
| | CGTGGATGAAGTTGGTGGTGAGGCCCTGGGCAGGTTGGTATCAAGGTTA |
| | CAAGACAGGTTTAAGGAGACCAATAGAAACTGGGCATGTGGAGACAGAG |
| | AAGACTCTTGGGTTTCTGATAGGCACTGACTCTCTCTGCCTATTGGTCTA |
| | TTTTCCCACCCTTAGGCTGCTGGTGGTCTACCCTTGGACCCAGAGGTTCT |
| | TTGAGTCCTTTGGGGATCTGTCCACTCCTGATGCTGTTATGGGCAACCCT |
| | AAGGTGAAGGCTCATGGCAAGAAAGTGCTCGGTGCCTTTAGTGATGGCC |
| | TGGCTCACCTGGACAACCTCAAGGGCACCTTTGCCACACTGAGTGAGCT |
| | GCACTGTGACAAGCTGC |

### Providing a First Probe Candidate Group

By the same method as in Example 1, probes were designed according to lengths (by length) (predetermined lengths: 17 to 35 mer) at each position from position 394, which is present 2 bp upstream from position 396 as the start position of the nucleotide mutation of interest (the C base), to position 397 as the end position of the nucleotide mutation of interest within the provided forward mutant target nucleic acid sequence, and these probes were provided as the first probe candidate group. In addition, probes were designed according to lengths (by length) (predetermined lengths: 17 to 35 mer) at each position from position 310, which is present 2 bp upstream from position 312 as the start position of the nucleotide mutation of interest (the G base), to position 313 as the end position of the nucleotide mutation of interest within the provided reverse mutant target nucleic acid sequence, and these probes were provided as the first probe candidate group. The number of probes designed for the forward and reverse mutant target nucleic acid sequences was 152, and of these, forward probes having 19 and 20 mer were summarized in Table 10 below.

TABLE 10

| Strand | Sequence | Start | End | Note |
|---|---|---|---|---|
| Forward | GACCTTCTCCTCAGGAGTC | 394 | 412 | SEQ ID NO: 35 |
| | GACCTTCTCCTCAGGAGTCA | 394 | 413 | SEQ ID NO: 36 |
| | ACCTTCTCCTCAGGAGTCA | 395 | 413 | SEQ ID NO: 37 |
| | ACCTTCTCCTCAGGAGTCAG | 395 | 414 | SEQ ID NO: 38 |
| | CCTTCTCCTCAGGAGTCAG | 396 | 414 | SEQ ID NO: 39 |
| | CCTTCTCCTCAGGAGTCAGA | 396 | 415 | SEQ ID NO: 40 |
| | CTTCTCCTCAGGAGTCAGA | 397 | 415 | SEQ ID NO: 41 |
| | CTTCTCCTCAGGAGTCAGAT | 397 | 416 | SEQ ID NO: 42 |

### Providing a Second Probe Candidate Group

By the same method and selection criteria as in Example 1, the second probe candidate group for the mutant target nucleic acid sequence were provided.

The number of probes satisfying the selection criterion among the probes included in the first probe candidate group was 43 and, of these, only five probes were summarized in Table 11 below.

TABLE 11

| Strand | Sequence | Start | End | Note |
|---|---|---|---|---|
| Forward | GACCTTCTCCTCAGGAGTCAGA | 394 | 415 | SEQ ID NO: 43 |
| | ACCTTCTCCTCAGGAGTCAGATG | 395 | 417 | SEQ ID NO: 44 |
| | ACCTTCTCCTCAGGAGTCAGATGCACCATGG | 395 | 425 | SEQ ID NO: 45 |
| | CCTTCTCCTCAGGAGTCAGATGC | 396 | 418 | SEQ ID NO: 46 |
| | CCTTCTCCTCAGGAGTCAGATGCACCATGGT | 396 | 426 | SEQ ID NO: 47 |

The 43 probes were arranged by giving ranks according to the arrangement criteria in Example 1.

### Providing a Third Probe Candidate Group

By the same method as in Example 1, out of probes having the same start position for designing a probe among the probes included in the second probe candidate group, the highest-ranked probes were provided as the third probe candidate group. Among the second probe candidate group comprising the 43 probes, five probes were selected as the third probe candidate group, and the results were summarized in Table 12 below.

TABLE 12

| Strand | Sequence | Note |
|---|---|---|
| Forward | GACCTTCTCCWCAGGAGTCAGRTGC | SEQ ID NO: 48 |
| Reverse | AAGGTCTGCCGTTACTGCCCTG | SEQ ID NO: 49 |
| Forward | ACCTTCTCCWCAGGAGTCAGRTGC | SEQ ID NO: 50 |
| Reverse | GGTCTGCCGTTACTGCCCTGT | SEQ ID NO: 51 |
| Forward | CCTTCTCCWCAGGAGTCAGRTGCAC | SEQ ID NO: 52 |

### Providing a Primer Candidate Group

By the same method, design conditions, and arrangement criteria as in Example 1, primer candidate group were provided.

As a result, 12287 primers were designed as the primer candidate group, and from primers having the same start position for designing a primer among the designed primers, the 190 highest-ranked primers were selected, and examples of the selected highest-ranked primers were summarized in Table 13 below.

TABLE 13

| Strand | Sequence | Position | Note |
|---|---|---|---|
| Forward | ACCCAAGAGTCTTCTCTGTIIIIICATGCCCAG | 253-285 | SEQ ID NO: 53 |
| Forward | TCTCTGTCTCCACATGCCIIIIITCTATTGGTC | 265-297 | SEQ ID NO: 54 |
| Forward | CATGCCCAGTTTCTATTGGTIIIIITAAACCTGTC | 277-311 | SEQ ID NO: 55 |
| Reverse | CCAGTGCCAGAAGAGCCIIIIICAGGTACGG | 82-112 | SEQ ID NO: 56 |
| Reverse | CTCCTAAGCCAGTGCCAGAIIIIICAAGGACAG | 74-106 | SEQ ID NO: 57 |
| Reverse | GAGGGTTTGAAGTCCAACIIIIIAGCCAGTGC | 57-88 | SEQ ID NO: 58 |

### Combinations of Probe and Primers

By the same method as in Example 1, combinations of a probe and primers were provided by combining the selected third probe candidate group and primer candidate group. Specifically, 10 combinations that produce 300-350 bp amplicons and have no dimer formed between a probe and a primer to be combined were created by combining the five probes and the 190 primers that had been selected, and out of these, three exemplary combinations were summarized in Table 14 below.

TABLE 14

| Combination | Type | Sequence | Note |
|---|---|---|---|
| 1 | Forward | ACACAACTGTGTTCACTAGIIIIITCAAACA | SEQ ID NO: 59 |
| | primer | GAC | |
| | Reverse | CTCCACATGCCCAGTTTIIIIIGGTCTCCTT | SEQ ID NO: 60 |
| | primer | | |
| | Probe | GACCTTCTCCWCAGGAGTCAGRTGC | SEQ ID NO: 48 |
| 2 | Forward | GCCAGGGCTGGGCATIIIIITCAGGGCAG | SEQ ID NO: 61 |
| | primer | | |
| | Reverse | CAGTTTCTATTGGTCTCCTTAAAIIIIICTTG | SEQ ID NO: 62 |
| | primer | TAACC | |
| | Probe | GACCTTCTCCWCAGGAGTCAGRTGC | SEQ ID NO: 48 |
| 3 | Forward | GGGCTGGGCATAAAAGTCIIIIIAGAGCCAT | SEQ ID NO: 63 |
| | primer | C | |
| | Reverse | TCTCCACATGCCCAGTTTIIIIIGGTCTCCTT | SEQ ID NO: 64 |
| | primer | | |
| | Probe | AAGGTCTGCCGTTACTGCCCTG | SEQ ID NO: 49 |

Having described a preferred embodiment of the present invention, it is to be understood that variants and modifications thereof falling within the spirit of the invention may become apparent to those skilled in this art, and the scope of this invention is to be determined by appended claims and their equivalents.

What is claimed is:

1. A computer-implemented method for preparing an oligonucleotide used to detect a nucleotide mutation of interest in a target nucleic acid sequence, comprising:
(a) inputting information about a wild-type target nucleic acid sequence and a nucleotide mutation of interest; wherein the wild-type target nucleic acid sequence is a target nucleic acid sequence not comprising the nucleotide mutation of interest, the information includes information about (i) the wild-type target nucleic acid sequence, (ii) a position of the nucleotide mutation of interest occurring in the wild-type target nucleic acid sequence and (iii) wild-type and mutant bases at the position of the nucleotide mutation of interest, and wherein the position of the nucleotide mutation of interest is expressed as a start position and an end position,
(b) providing the wild-type target nucleic acid sequence and a mutant target nucleic acid sequence by using the input information; wherein the mutant target nucleic acid sequence is a target nucleic acid sequence comprising the nucleotide mutation of interest, and the wild-type and mutant target nucleic acid sequences include forward and reverse wild-type and mutant target nucleic acid sequences, respectively,
(c) providing a first oligonucleotide candidate group for the mutant target nucleic acid sequence by designing oligonucleotides used to detect the nucleotide mutation of interest in a predetermined region comprising the nucleotide mutation of interest within the mutant target nucleic acid sequence;
(d) providing oligonucleotides satisfying the following selection criteria as a second oligonucleotide candidate group for the mutant target nucleic acid sequence by analyzing matching of the wild-type target nucleic acid sequence with the oligonucleotides included in the first oligonucleotide candidate group for the mutant target nucleic acid sequence; wherein the selection criteria include that (i) the number of mismatches between the wild-type target nucleic acid sequence and a predetermined region at the 5′-end, middle or 3′-end of an oligonucleotide is one or more; or (ii) the ratio of mismatches between the wild-type target nucleic acid sequence and an oligonucleotide is a predetermined value or more; and
(e) providing a third oligonucleotide candidate group by selecting oligonucleotides from the second oligonucleotide candidate group for the mutant target nucleic acid sequence; wherein the third oligonucleotide candidate group is used to detect the nucleotide mutation of interest in the target nucleic acid sequence.

2. The method according to claim 1, wherein the nucleotide mutation of interest is a substitution, an inversion, an insertion, a deletion, a duplication, or a combination thereof.

3. The method according to claim 1, wherein the oligonucleotide is a probe and/or a primer.

4. The method according to claim 1, wherein the wild-type target nucleic acid sequence in step (a) has a predetermined length comprising a position of the nucleotide mutation of interest.

5. The method according to claim 1, wherein the oligonucleotides in step (c) are designed to have matching or complementary sequences to the predetermined region comprising the nucleotide mutation of interest within the mutant target nucleic acid sequence.

6. The method according to claim 1, wherein the oligonucleotides in step (c) are designed according to lengths at positions from a predetermined position upstream of the start position of the nucleotide mutation of interest within the mutant target nucleic acid sequence to the end position thereof.

7. The method according to claim 1, wherein the oligonucleotides in step (c) are designed to satisfy at least one of the following conditions:
(i) a length of 10-60 nucleotides;
(ii) a Tm value of 50-85° C.;
(iii) exclusion of a G-run sequence with at least three Gs; and
(iv) a GC content of 40% or more in the 5′-end portion.

8. The method according to claim 1, wherein the mismatches (i) in step (d) are minimum mismatches between the wild-type target nucleic acid sequence and the predetermined region of the 5′-end, middle, or 3′-end of the oligonucleotide, and the mismatches (ii) in step (d) are minimum mismatches between the wild-type target nucleic acid sequence and the oligonucleotide.

9. The method according to claim 1, wherein the method further comprises, after step (d), d-1) arranging the oligonucleotides included in the second oligonucleotide candidate group by giving ranks according to at least one of the following arrangement criteria:
(i) the position of a mismatch in an oligonucleotide as a result of the matching analysis in step (d); wherein the closer the position of the mismatch is to the 5′-end, 3′-end, or middle, the higher the rank,
(ii) the number of mismatches of an oligonucleotide as a result of the matching analysis in step (d); wherein the larger the number of the mismatches, the higher the rank,
(iii) a Tm value of an oligonucleotide; wherein the higher the Tm value, the higher the rank,
(iv) a GC content in a predetermined region of the 5′-end, middle, or 3′-end of an oligonucleotide; wherein the higher the GC content, the higher the rank,
(v) the number of consecutive G bases included in an oligonucleotide; wherein the smaller the number of consecutive G bases, the higher the rank,
(vi) the number or proportion of consecutive nucleotides involved in the formation of a homodimer when the oligonucleotide forms the homodimer; wherein the smaller the number or proportion, the higher the rank,
(vii) a hairpin structure-forming free energy value (ΔG value); wherein the larger the free energy value, the higher the rank, and
(viii) a length; wherein the shorter the length, the higher the rank.

10. The method according to claim 1, wherein the third oligonucleotide candidate group in step (e) is selected by a method comprising the following steps:
(e-1) arranging by giving ranks according to at least one of the following arrangement criteria to oligonucleotides having the same start position for designing an oligonucleotide among the oligonucleotides included in the second oligonucleotide candidate group; and
(e-2) selecting the highest ranked oligonucleotide from oligonucleotides having the same start position for designing the arranged oligonucleotides, and wherein the arrangement criteria include the following:
(i) the position of a mismatch in an oligonucleotide as a result of the matching analysis in step (d); wherein the closer the position of the mismatch is to the 5′-end, 3′-end, or middle, the higher the rank,
(ii) the number of mismatches in an oligonucleotide as a result of the matching analysis in step (d); wherein the larger the number of the mismatches, the higher the rank,
(iii) a Tm value of an oligonucleotide; wherein the higher the Tm value, the higher the rank,
(iv) a GC content in a predetermined region of the 5′-end, middle, or 3′-end of an oligonucleotide; wherein the higher the GC content, the higher the rank,
(v) the number of consecutive G bases included in an oligonucleotide; wherein the smaller the number of consecutive G bases, the higher the rank,
(vi) the number or proportion of consecutive nucleotides involved in the formation of a homodimer when the oligonucleotide forms the homodimer; wherein the smaller the number or proportion, the higher the rank,
(vii) a hairpin structure-forming free energy value (ΔG value); wherein the larger the free energy value, the higher the rank, and
(viii) a length; wherein the shorter the length, the higher the rank.

11. The method according to claim 1, wherein the first to third oligonucleotide candidate groups are first to third probe candidate groups, respectively, and the method further comprises, after step (e), the following steps:
(f) providing a primer candidate group for the mutant target nucleic acid sequence by designing primers to amplify a predetermined region comprising the nucleotide mutation of interest within the mutant target nucleic acid sequence; and
(g) providing a combination of a probe and primers by combining the third probe candidate group and the primer candidate group.

12. The method according to claim 11, wherein the primers in step (f) are designed to satisfy at least one of the following conditions:
(i) a Tm value of 40-70° C.;
(ii) a length of 15-50 bp nucleotides; and
(iii) exclusion of a G-run sequence with at least five Gs.

13. The method according to claim 11, wherein the method further comprises, after step (f), the following steps:
(f-1) arranging by giving ranks according to at least one of the following arrangement criteria to primers having the same start position for designing a primer among the primers included in the primer candidate group; and
(f-2) selecting the highest ranked primer from primers having the same start position for designing the arranged primers, and wherein the arrangement criteria include the following:
(i) the number or proportion of use of a degenerate base and/or universal base introduced into a primer; wherein the smaller the number or proportion of use, the higher the rank,
(ii) the number of primer patterns generated by the introduction of a degenerate base; wherein the smaller the number of patterns, the higher the rank,
(iii) the number of (A)ₙ, (T)ₙ, or (C)ₙ mononucleotide run sequences; wherein the smaller the number of the sequences, the higher the rank,
(iv) the number or proportion of consecutive nucleotides involved in the formation of a homodimer when a primer forms the homodimer; wherein the smaller the number or proportion, the higher the rank,
(v) a hairpin structure-forming free energy value (ΔG value); wherein the larger the free energy value, the higher the rank,
(vi) a Tm value; wherein the higher the Tm value, the higher the rank,
(vii) a GC content; wherein the more the GC content, the higher the rank, and
(viii) a length; wherein the shorter the length, the higher the rank.

14. A computer readable storage medium containing instructions to configure a processor to perform a method for preparing an oligonucleotide used to detect a nucleotide mutation of interest in a target nucleic acid sequence, the method comprising:
(a) inputting information about a wild-type target nucleic acid sequence and a nucleotide mutation of interest; wherein the wild-type target nucleic acid sequence is a target nucleic acid sequence not comprising the nucleotide mutation of interest, the information includes information about (i) the wild-type target nucleic acid sequence, (ii) a position of the nucleotide mutation of interest occurring in the wild-type target nucleic acid sequence and (iii) wild-type and mutant bases at the position of the nucleotide mutation of interest, and wherein the position of the nucleotide mutation of interest is expressed as a start position and an end position,
(b) providing the wild-type target nucleic acid sequence and a mutant target nucleic acid sequence by using the input information; wherein the mutant target nucleic acid sequence is a target nucleic acid sequence comprising the nucleotide mutation of interest, and the wild-type and mutant target nucleic acid sequences include forward and reverse wild-type and mutant target nucleic acid sequences, respectively,
(c) providing a first oligonucleotide candidate group for the mutant target nucleic acid sequence by designing oligonucleotides used to detect the nucleotide mutation of interest in a predetermined region comprising the nucleotide mutation of interest within the mutant target nucleic acid sequence;
(d) providing oligonucleotides satisfying the following selection criteria as a second oligonucleotide candidate group for the mutant target nucleic acid sequence by analyzing matching of the wild-type target nucleic acid sequence with the oligonucleotides included in the first oligonucleotide candidate group for the mutant target nucleic acid sequence; wherein the selection criteria include that (i) the number of mismatches between the wild-type target nucleic acid sequence and a predetermined region at the 5′-end, middle or 3′-end of an oligonucleotide is one or more; or (ii) the ratio of mismatches between the wild-type target nucleic acid sequence and an oligonucleotide is a predetermined value or more; and
(e) providing a third oligonucleotide candidate group by selecting oligonucleotides from the second oligonucleotide candidate group for the mutant target nucleic acid sequence; wherein the third oligonucleotide candidate group is used to detect the nucleotide mutation of interest in the target nucleic acid sequence.


SEQUENCE LISTING (64 sequences)

SEQ ID NO: 1 - DNA, 20 bp, Artificial Sequence
  Synthetic nucleotide_wild-type target nucleic acid sequence
cagatctgttttaaacgact

SEQ ID NO: 2 - DNA, 21 bp, Artificial Sequence
  Synthetic nucleotide_mutant target nucleic acid sequence
cagatctgtttttaaacgact

SEQ ID NO: 3 - DNA, 12 bp, Artificial Sequence
  Synthetic nucleotide_oligonucleotide for insertion mutation
ttttaaacgact

SEQ ID NO: 4 - DNA, 1139 bp, Homo sapiens
  gene (1)..(1139): Forward wild-type target nucleic acid sequence of ApoE gene
ctctgtccttccctagctcttttatatagagacagagagatggggtctcactgtgttgcccaggctggtcttgaacttctgggctcaagcgatcctcccgcctcggcctcccaaagtgctgggattagaggcatgagccaccttgcccggcctcctagctccttcttcgtctctgcctctgccctctgcatctgctctctgcatctgtctctgtctccttctctcggcctctgccccgttccttctctccctcttgggtctctctggctcatccccatctcgcccgccccatcccagcccttctccccgcctcccactgtgcgacaccctcccgccctctcggccgcagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggag

SEQ ID NO: 5 - DNA, 1139 bp, Homo sapiens
  gene (1)..(1139): Reverse wild-type target nucleic acid sequence of ApoE gene
ctcccgctgcaggctgcgcggaggcaggaggcacggggtggcgtggggtcgcatggctgcaggcttcggcgttcagtgattgtcgctgggcacaggggcggcgctggtgcccacggcagcctgcaccttctccaccagcccggcccactggcgctgcatgtcttccaccaggggctcgaaccagctcttgaggcgggcctggaaggcctcggcctgcaggcgtatctgctgggcctgctcctccagcttggcgcgcacctccgccacctgctccttcacctcgtccaggcggtcgcgggtccggctgcccatctcctccatccgcgcgcgcagccgctcgccccaggcctgggcccgctcctgtagcggctggccggccagggagcccacagtggcggcccgcacgcggccctgttccaccaggggccccaggcgctcgcggatggcgctgaggccgcgctcggcgccctcgcgggccccggcctggtacactgccaggcgcttctgcaggtcatcggcatcgcggaggagccgcttacgcagcttgcgcaggtgggaggcgaggcgcacccgcagctcctcggtgctctggccgagcatggcctgcacctcgccgcggtactgcaccaggcggccgcacacgtcctccatgtccgcgcccagccgggcctgcgccgcctgcagctccttggacagccgtgcccgcgtctcctccgccaccggggtcagttgttcctccagttccgatttgtaggccttcaactccttcatggtctcgtccatcagcgccctgcggccgagagggcgggagggtgtcgcacagtgggaggcggggagaagggctgggatggggcgggcgagatggggatgagccagagagacccaagagggagagaaggaacggggcagaggccgagagaaggagacagagacagatgcagagagcagatgcagagggcagaggcagagacgaagaaggagctaggaggccgggcaaggtggctcatgcctctaatcccagcactttgggaggccgaggcgggaggatcgcttgagcccagaagttcaagaccagcctgggcaacacagtgagaccccatctctctgtctctatataaaagagctagggaaggacagag

SEQ ID NO: 6 - DNA, 1139 bp, Homo sapiens
  gene (1)..(1139): Forward mutant target nucleic acid sequence of ApoE gene
ctctgtccttccctagctcttttatatagagacagagagatggggtctcactgtgttgcccaggctggtcttgaacttctgggctcaagcgatcctcccgcctcggcctcccaaagtgctgggattagaggcatgagccaccttgcccggcctcctagctccttcttcgtctctgcctctgccctctgcatctgctctctgcatctgtctctgtctccttctctcggcctctgccccgttccttctctccctcttgggtctctctggctcatccccatctcgcccgccccatcccagcccttctccccgcctcccactgtgcgacaccctcccgccctctcggccgcagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgcgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggag

SEQ ID NO: 7 - DNA, 1139 bp, Homo sapiens
  gene (1)..(1139): Reverse mutant target nucleic acid sequence of ApoE gene
ctcccgctgcaggctgcgcggaggcaggaggcacggggtggcgtggggtcgcatggctgcaggcttcggcgttcagtgattgtcgctgggcacaggggcggcgctggtgcccacggcagcctgcaccttctccaccagcccggcccactggcgctgcatgtcttccaccaggggctcgaaccagctcttgaggcgggcctggaaggcctcggcctgcaggcgtatctgctgggcctgctcctccagcttggcgcgcacctccgccacctgctccttcacctcgtccaggcggtcgcgggtccggctgcccatctcctccatccgcgcgcgcagccgctcgccccaggcctgggcccgctcctgtagcggctggccggccagggagcccacagtggcggcccgcacgcggccctgttccaccaggggccccaggcgctcgcggatggcgctgaggccgcgctcggcgccctcgcgggccccggcctggtacactgccaggcgcttctgcaggtcatcggcatcgcggaggagccgcttacgcagcttgcgcaggtgggaggcgaggcgcacccgcagctcctcggtgctctggccgagcatggcctgcacctcgccgcggtactgcaccaggcggccgcgcacgtcctccatgtccgcgcccagccgggcctgcgccgcctgcagctccttggacagccgtgcccgcgtctcctccgccaccggggtcagttgttcctccagttccgatttgtaggccttcaactccttcatggtctcgtccatcagcgccctgcggccgagagggcgggagggtgtcgcacagtgggaggcggggagaagggctgggatggggcgggcgagatggggatgagccagagagacccaagagggagagaaggaacggggcagaggccgagagaaggagacagagacagatgcagagagcagatgcagagggcagaggcagagacgaagaaggagctaggaggccgggcaaggtggctcatgcctctaatcccagcactttgggaggccgaggcgggaggatcgcttgagcccagaagttcaagaccagcctgggcaacacagtgagaccccatctctctgtctctatataaaagagctagggaaggacagag

SEQ ID NO: 8 - DNA, 19 bp, Artificial Sequence
  Synthetic nucleotide_Forward probe for mutant target nucleic acid sequence of ApoE gene
tgcgcggccgcctggtgca

SEQ ID NO: 9 - DNA, 20 bp, Artificial Sequence
  Synthetic nucleotide_Forward probe for mutant target nucleic acid sequence of ApoE gene
tgcgcggccgcctggtgcag

SEQ ID NO: 10 - DNA, 19 bp, Artificial Sequence
  Synthetic nucleotide_Forward probe for mutant target nucleic acid sequence of ApoE gene
gcgcggccgcctggtgcag

SEQ ID NO: 11 - DNA, 20 bp, Artificial Sequence
  Synthetic nucleotide_Forward probe for mutant target nucleic acid sequence of ApoE gene
gcgcggccgcctggtgcagt

SEQ ID NO: 12 - DNA, 19 bp, Artificial Sequence
  Synthetic nucleotide_Forward probe for mutant target nucleic acid sequence of ApoE gene
cgcggccgcctggtgcagt

SEQ ID NO: 13 - DNA, 20 bp, Artificial Sequence
  Synthetic nucleotide_Forward probe for mutant target nucleic acid sequence of ApoE gene
cgcggccgcctggtgcagta

SEQ ID NO: 14 - DNA, 17 bp, Artificial Sequence
  Synthetic nucleotide_Forward probe for mutant target nucleic acid sequence of ApoE gene
cgcggccgcctggtgca

SEQ ID NO: 15 - DNA, 18 bp, Artificial Sequence
  Synthetic nucleotide_Forward probe for mutant target nucleic acid sequence of ApoE gene
cgcggccgcctggtgcag

SEQ ID NO: 16 - DNA, 19 bp, Artificial Sequence
  Synthetic nucleotide_Forward probe for mutant target nucleic acid sequence of ApoE gene
cgcggccgcctggtgcagt

SEQ ID NO: 17 - DNA, 20 bp, Artificial Sequence
  Synthetic nucleotide_Forward probe for mutant target nucleic acid sequence of ApoE gene
cgcggccgcctggtgcagta

SEQ ID NO: 18 - DNA, 21 bp, Artificial Sequence
  Synthetic nucleotide_Forward probe for mutant target nucleic acid sequence of ApoE gene
cgcggccgcctggtgcagtac

SEQ ID NO: 19 - DNA, 19 bp, Artificial Sequence
  Synthetic nucleotide_Reverse probe for mutant target nucleic acid sequence of ApoE gene
gcacgtcctccatgtccgc

SEQ ID NO: 20 - DNA, 30 bp, Artificial Sequence
  Synthetic nucleotide_Forward primer for mutant target nucleic acid sequence of ApoE gene
  misc_feature (17)..(21): n is deoxyinosine
tctctcggcctctgccnnnnnccttctctc

SEQ ID NO: 21 - DNA, 31 bp, Artificial Sequence
  Synthetic nucleotide_Forward primer for mutant target nucleic acid sequence of ApoE gene
  misc_feature (18)..(22): n is deoxyinosine
atcccagcccttctcccnnnnncccactgtg

SEQ ID NO: 22 - DNA, 31 bp, Artificial Sequence
  Synthetic nucleotide_Forward primer for mutant target nucleic acid sequence of ApoE gene
  misc_feature (18)..(22): n is deoxyinosine
tggacgagaccatgaagnnnnngaaggccta

SEQ ID NO: 23 - DNA, 29 bp, Artificial Sequence
  Synthetic nucleotide_Reverse primer for mutant target nucleic acid sequence of ApoE gene
  misc_feature (16)..(20): n is deoxyinosine
gtgggaggcgaggcgnnnnngcagctcct

SEQ ID NO: 24 - DNA, 30 bp, Artificial Sequence
  Synthetic nucleotide_Reverse primer for mutant target nucleic acid sequence of ApoE gene
  misc_feature (17)..(21): n is deoxyinosine
gcagctcctcggtgctnnnnncgagcatgg

SEQ ID NO: 25 - DNA, 29 bp, Artificial Sequence
  Synthetic nucleotide_Reverse primer for mutant target nucleic acid sequence of ApoE gene
  misc_feature (16)..(20): n is deoxyinosine
tggcctgcacctcgcnnnnntactgcacc

SEQ ID NO: 26 - DNA, 29 bp, Artificial Sequence
  Synthetic nucleotide_Forward primer for mutant target nucleic acid sequence of ApoE gene
  misc_feature (16)..(20): n is deoxyinosine
gcagggcgctgatggnnnnnaccatgaag

SEQ ID NO: 27 - DNA, 30 bp, Artificial Sequence
  Synthetic nucleotide_Reverse primer for mutant target nucleic acid sequence of ApoE gene
  misc_feature (17)..(21): n is deoxyinosine
cagcccttctccccgcnnnnnactgtgcga

SEQ ID NO: 28 - DNA, 31 bp, Artificial Sequence
  Synthetic nucleotide_Forward primer for mutant target nucleic acid sequence of ApoE gene
  misc_feature (18)..(22): n is deoxyinosine
catcccagcccttctccnnnnntcccactgt

SEQ ID NO: 29 - DNA, 29 bp, Artificial Sequence
  Synthetic nucleotide_Reverse primer for mutant target nucleic acid sequence of ApoE gene
  misc_feature (16)..(20): n is deoxyinosine
tgggaggcgaggcgcnnnnncagctcctc

SEQ ID NO: 30 - DNA, 29 bp, Artificial Sequence
  Synthetic nucleotide_Forward primer for mutant target nucleic acid sequence of ApoE gene
  misc_feature (16)..(20): n is deoxyinosine
ccgcagggcgctgatnnnnnagaccatga

SEQ ID NO: 31 - DNA, 707 bp, Homo sapiens
  gene (1)..(707): Forward wild-type target nucleic acid sequence of HBB gene
gcagcttgtcacagtgcagctcactcagtgtggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcctcaggagtcagatgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctact

SEQ ID NO: 32 - DNA, 707 bp, Homo sapiens
  gene (1)..(707): Reverse wild-type target nucleic acid sequence of HBB gene
agtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgc

SEQ ID NO: 33 - DNA, 708 bp, Homo sapiens
  gene (1)..(708): Forward mutant target nucleic acid sequence of HBB gene
gcagcttgtcacagtgcagctcactcagtgtggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagaccttctcctcaggagtcagatgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctact

SEQ ID NO: 34 - DNA, 708 bp, Homo sapiens
  gene (1)..(708): Reverse mutant target nucleic acid sequence of HBB gene
agtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcatctgactcctgaggagaaggtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgc

SEQ ID NO: 35 - DNA, 19 bp, Artificial Sequence
  Synthetic nucleotide_Forward probe for mutant target nucleic acid sequence of HBB gene
gaccttctcctcaggagtc

SEQ ID NO: 36 - DNA, 20 bp, Artificial Sequence
  Synthetic nucleotide_Forward probe for mutant target nucleic acid sequence of HBB gene
gaccttctcctcaggagtca

SEQ ID NO: 37 - DNA, 19 bp, Artificial Sequence
  Synthetic nucleotide_Forward probe for mutant target nucleic acid sequence of HBB gene
accttctcctcaggagtca

SEQ ID NO: 38 - DNA, 20 bp, Artificial Sequence
  Synthetic nucleotide_Forward probe for mutant target nucleic acid sequence of HBB gene
accttctcctcaggagtcag

SEQ ID NO: 39 - DNA, 19 bp, Artificial Sequence
  Synthetic nucleotide_Forward probe for mutant target nucleic acid sequence of HBB gene
ccttctcctcaggagtcag

SEQ ID NO: 40 - DNA, 20 bp, Artificial Sequence
  Synthetic nucleotide_Forward probe for mutant target nucleic acid sequence of HBB gene
ccttctcctcaggagtcaga

SEQ ID NO: 41 - DNA, 19 bp, Artificial Sequence
  Synthetic nucleotide_Forward probe for mutant target nucleic acid sequence of HBB gene
cttctcctcaggagtcaga

SEQ ID NO: 42 - DNA, 20 bp, Artificial Sequence
  Synthetic nucleotide_Forward probe for mutant target nucleic acid sequence of HBB gene
cttctcctcaggagtcagat

SEQ ID NO: 43 - DNA, 22 bp, Artificial Sequence
  Synthetic nucleotide_Forward probe for mutant target nucleic acid sequence of HBB gene
gaccttctcctcaggagtcaga

SEQ ID NO: 44 - DNA, 23 bp, Artificial Sequence
  Synthetic nucleotide_Forward probe for mutant target nucleic acid sequence of HBB gene
accttctcctcaggagtcagatg

SEQ ID NO: 45 - DNA, 31 bp, Artificial Sequence
  Synthetic nucleotide_Forward probe for mutant target nucleic acid sequence of HBB gene
accttctcctcaggagtcagatgcaccatgg

SEQ ID NO: 46 - DNA, 23 bp, Artificial Sequence
  Synthetic nucleotide_Forward probe for mutant target nucleic acid sequence of HBB gene
ccttctcctcaggagtcagatgc

SEQ ID NO: 47 - DNA, 31 bp, Artificial Sequence
  Synthetic nucleotide_Forward probe for mutant target nucleic acid sequence of HBB gene
ccttctcctcaggagtcagatgcaccatggt

SEQ ID NO: 48 - DNA, 25 bp, Artificial Sequence
  Synthetic nucleotide_Forward probe for mutant target nucleic acid sequence of HBB gene
gaccttctccwcaggagtcagrtgc

SEQ ID NO: 49 - DNA, 22 bp, Artificial Sequence
  Synthetic nucleotide_Reverse probe for mutant target nucleic acid sequence of HBB gene
aaggtctgccgttactgccctg

SEQ ID NO: 50 - DNA, 24 bp, Artificial Sequence
  Synthetic nucleotide_Forward probe for mutant target nucleic acid sequence of HBB gene
accttctccwcaggagtcagrtgc

SEQ ID NO: 51 - DNA, 21 bp, Artificial Sequence
  Synthetic nucleotide_Reverse probe for mutant target nucleic acid sequence of HBB gene
ggtctgccgttactgccctgt

SEQ ID NO: 52 - DNA, 25 bp, Artificial Sequence
  Synthetic nucleotide_Forward probe for mutant target nucleic acid sequence of HBB gene
ccttctccwcaggagtcagrtgcac

SEQ ID NO: 53 - DNA, 33 bp, Artificial Sequence
  Synthetic nucleotide_Forward primer for mutant target nucleic acid sequence of HBB gene
  misc_feature (20)..(24): n is deoxyinosine
acccaagagtcttctctgtnnnnncatgcccag

SEQ ID NO: 54 - DNA, 33 bp, Artificial Sequence
  Synthetic nucleotide_Forward primer for mutant target nucleic acid sequence of HBB gene
  misc_feature (19)..(23): n is deoxyinosine
tctctgtctccacatgccnnnnntctattggtc

SEQ ID NO: 55 - DNA, 35 bp, Artificial Sequence
  Synthetic nucleotide_Forward primer for mutant target nucleic acid sequence of HBB gene
  misc_feature (21)..(25): n is deoxyinosine
catgcccagtttctattggtnnnnntaaacctgtc

SEQ ID NO: 56 - DNA, 31 bp, Artificial Sequence
  Synthetic nucleotide_Reverse primer for mutant target nucleic acid sequence of HBB gene
  misc_feature (18)..(22): n is deoxyinosine
ccagtgccagaagagccnnnnncaggtacgg

SEQ ID NO: 57 - DNA, 33 bp, Artificial Sequence
  Synthetic nucleotide_Reverse primer for mutant target nucleic acid sequence of HBB gene
  misc_feature (20)..(24): n is deoxyinosine
ctcctaagccagtgccagannnnncaaggacag

SEQ ID NO: 58 - DNA, 32 bp, Artificial Sequence
  Synthetic nucleotide_Reverse primer for mutant target nucleic acid sequence of HBB gene
  misc_feature (19)..(23): n is deoxyinosine
gagggtttgaagtccaacnnnnnagccagtgc

SEQ ID NO: 59 - DNA, 34 bp, Artificial Sequence
  Synthetic nucleotide_Forward primer for mutant target nucleic acid sequence of HBB gene
  misc_feature (20)..(24): n is deoxyinosine
acacaactgtgttcactagnnnnntcaaacagac

SEQ ID NO: 60 - DNA, 31 bp, Artificial Sequence
  Synthetic nucleotide_Reverse primer for mutant target nucleic acid sequence of HBB gene
  misc_feature (18)..(22): n is deoxyinosine
ctccacatgcccagtttnnnnnggtctcctt

SEQ ID NO: 61 - DNA, 29 bp, Artificial Sequence
  Synthetic nucleotide_Forward primer for mutant target nucleic acid sequence of HBB gene
  misc_feature (16)..(20): n is deoxyinosine
gccagggctgggcatnnnnntcagggcag

SEQ ID NO: 62 - DNA, 37 bp, Artificial Sequence
  Synthetic nucleotide_Reverse primer for mutant target nucleic acid sequence of HBB gene
  misc_feature (24)..(28): n is deoxyinosine
cagtttctattggtctccttaaannnnncttgtaacc

SEQ ID NO: 63 - DNA, 32 bp, Artificial Sequence
  Synthetic nucleotide_Forward primer for mutant target nucleic acid sequence of HBB gene
  misc_feature (19)..(23): n is deoxyinosine
gggctgggcataaaagtcnnnnnagagccatc

SEQ ID NO: 64 - DNA, 32 bp, Artificial Sequence
  Synthetic nucleotide_Reverse primer for mutant target nucleic acid sequence of HBB gene
  misc_feature (19)..(23): n is deoxyinosine
tctccacatgcccagtttnnnnnggtctcctt